(19) United States
(12) Patent Application Publication
(10) Pub. No.: US 2012/0058084 A1
(43) Pub. Date: Mar. 8, 2012

(54) Interferon Alpha Carrier Prodrugs

(75) Inventors: Harald Rau, Heidelberg (DE); Silvia Kaden-Vagt, Heidelberg (DE)

(73) Assignee: ASCENDIS PHARMA AS, Hellerup (DK)

(21) Appl. No.: 13/254,869

(22) PCT Filed: Mar. 4, 2010
(86) PCT No.: PCT/EP2010/052745

§ 371 Date: Nov. 16, 2011

(30) Foreign Application Priority Data
Mar. 5, 2009 (EP) 09154449.4
Dec. 22, 2009 (EP) 09180477.3

(51) Int. Cl. A61K 38/21; A61P 31/12; B67D 7/74; C07K 14/56

(52) U.S. Cl. 424/85.7; 530/351; 222/129; 222/1

(57) ABSTRACT

The present invention relates to a pharmaceutical composition comprising a water-soluble polymeric carrier linked prodrug of interferon alpha, wherein the prodrug is capable of releasing free interferon alpha, wherein the release half life under physiological conditions is at least 4 days. The invention further relates to prodrugs for said pharmaceutical composition and their use for treating, controlling, delaying or preventing a condition that can benefit from interferon alpha treatment, such as hepatitis C.

The present invention relates to a pharmaceutical composition comprising water-soluble polymeric carrier linked prodrugs of interferon alpha and their use for treating, controlling, delaying or preventing a condition that can benefit from interferon alpha treatment, such as hepatitis C.

Interferons were first described more than 50 years ago by Isaacs and Lindenmann in 1957, when they discovered that a factor was released when heat inactivated influenza virus was incubated with chick embryo cells, inducing resistance to infection with homologous or heterologous viruses. The scientific community remained skeptic of this interfering factor, due to unsuccessful purification and isolation. It was not until 1980 when cloning of the interferon molecules became possible, that the pleiotropic properties of the interferons became fully acknowledged.

Interferons are now considered central mediators of the immune response, and are attributed three major biological activities: antiviral activity, anti-proliferative activity and immunoregulatory activity.

Classification of interferons is based on sequence, chromosomal location and receptor specificity. Interferon alpha (interferon-α) and interferon beta (interferon-β)are the most predominant Type I interferons and transmit signals through a receptor complex composed of two subunits IFNAR-1 and IFNAR-2. Also included in the Type I group of interferons is consensus interferon, interferon alfacon-1.

In the early 1980s experiments with radiolabelled interferon led to the conclusion that there are specific high-affinity cell-surface receptors, which are distinct for type I and type II interferon.

Interferon alpha binds to the afore-mentioned dimeric receptor. The production of interferon alpha is induced by exposure to double stranded RNA (dsRNA) from viruses. At some point in their replication most viruses produce dsRNA, which is a potent inducer of interferon alpha that in turn mediates the immune response. The nature of the immune response is not fully understood, but it is known that interferon alpha induces an antiviral state at the cellular level, whereby the replication of virus is impaired through induction of a number of antiviral proteins.

The symptoms associated with viral infections, can be replicated by administration of interferon alpha to volunteers. Therefore the flu-like adverse effects associated with interferon alpha treatment is believed to be of similar nature as the flu-like symptoms associated with viral infections is also caused by endogenous interferon alpha production.

Interferon alpha is widely used to treat hepatitis C. A major goal is to reduce complications associated with chronic hepatitis C infection. This is principally achieved by eradicating the virus. Accordingly treatment response can be measured as the results of hepatitis C RNA testing. The goal is to achieve sustained viral response (SVR) which is defined as undetectable hepatitis C RNA in the serum 6 month after the end of treatment.

Interferon alpha monotherapy was until recently the only treatment option for chronic hepatitis C. Three interferon alpha compounds are used in hepatitis C treatment, namely interferon-α2a, interferon-α2b, and a recombinant non-naturally occurring type-I interferon consisting of 166-amino acid sequence with 88% homology with interferon-α2b commercialized as Infergen®.

When used as monotherapy interferon alpha initially reduces hepatitis C RNA levels in 50-60% of the patients, but a sustained viral response is only achieved in 10-20% of patients. The remaining patients relapse and develop symptoms of active hepatitis C again. Because of this low level of treatment success, interferon alpha therapy is combined with ribavirin. Ribavirin is a nucleoside analog-like compound that displays antiviral activity against a range of viruses. The synergistic effect observed with interferon alpha is not clearly understood, but several clinical trials have shown the superiority of combination therapy of interferon alpha and ribavirin compared to interferon alpha monotherapy.

Interferon alpha is rapidly eliminated in patients, which reduces its antiviral efficacy. Several mechanisms are involved in the elimination of interferon alpha, including proteolytic degradation, renal clearance and receptor mediated clearance. For this reason interferon alpha requires frequent administration to patients in order to achieve a sustained anti-viral response. Unconjugated interferon alpha is administered 3 times a week, which still does not ensure full interferon coverage throughout therapy. Constant antiviral pressure is important to prevent replication and the emergence of resistant variants. Furthermore, the short plasma half life results in large peak-to-trough ratios, which translate into increased adverse effects, such as the flu-like symptoms commonly associated with interferon alpha therapy is prominent at high plasma concentrations.

In order to develop a more effective interferon alpha therapy which exerts constant antiviral pressure, PEGylated versions of interferon alpha have been developed and approved for hepatitis C treatment, namely Pegasys and PEGIntron. Permanent conjugation of a poly ethylene glycol (PEG) moiety to the interferon alpha protein has enabled a significant increase of the plasma half life, allowing once weekly administration. PEGylation of interferon alpha increases plasma half life by reducing glomerular filtration, proteolysis and receptor mediated clearance. In addition, pegylation may decrease adverse events caused by large variations in peak-to-trough ratios (P. Caliceti, Digestive and Liver Disease 36 Suppl. 3 (2004), S334-S339).

A major drawback of this pegylation technology is a reduced bioactivity of the PEG conjugated protein. In the case of conjugation of interferon-α2a with a branched 40 kDa PEG only 7% of the bioactivity of the unconjugated protein is retained. This necessitates administration of higher doses of PEG-interferon-α2a conjugate (P. Bailon et al., Bioconjugate Chem. 2001, 12, 195-202). Furthermore, attachment of large PEG molecules restricts the conjugate primarily to the blood volume, and hence prevents the conjugate of penetrating all target tissues, resulting in decreased volume of distribution. Thus, viral reservoirs outside the plasma are not targeted, which is likely to play a role in the persistence and reactivation of the hepatitis C infection.

Hepatitis C is known to infect different extrahepatic sites such as peripheral blood mononuclear cells (PBMCs), renal cells, thyroid cells, and gastric cells, and evidence suggests that these could represent replicative compartments for the virus. Therefore, reaching therapeutic relevant concentrations in these extrahepatic viral pools is likely to be important for preventing virologic relapse and re-infection of hepatocytes. Currently, the low volume of distribution of permanently PEGylated interferon alpha conjugates strongly indicate that these compounds are not reaching these compartments, which most likely results in the relative high viral relapse rates that were observed after treatment with these compounds.

Different approaches have been tried to solve these problems. One of the marketed Peg-interferons, PEGIntron has a larger volume of distribution than Pegasys, partly due to a smaller PEG moiety (12 kDa versus 40 kDa) and partial pegylation at HIS³⁴ which is unstable and releases free interferon-α2b in vivo. The volume of distribution for PEGIntron is approximately 30% smaller than that of unconjugated interferon-α2b. (P. Caliceti, Digestive and Liver Disease 36 Suppl. 3 (2004), S334-S339). Initial results from the IDEAL clinical trial suggest that the larger volume of distribution of PEGIntron as compared to Pegasys in fact translates into lower relapse rates. (company web site, http://www.schering-plough.com).

The half life of PEGIntron® of about 40-58 hours is significantly shorter than that of Pegasys (half life 160 hours), resulting in large peak-to-trough ratio and suboptimal antiviral pressure when administered once weekly.

Addition of a polymeric carrier like a PEG molecule to the interferon introduces the problem of injection site reactions. Following administration of standard doses of pegylated interferon-α and ribavirin up to 58% of patients on Pegasys experience injection site reactions. For PEGIntron the incidence is 36% (Russo and Fried, Gastroenterology 2003; 124: 1711-1719). When administering unconjugated interferon-α2b only 5% of hepatitis C patients experience injection site reactions (Intron A prescribing information). Based on this it appears that the incidence of injection site reactions is influenced by both residual activity and residence time of the pegylated interferon-α. Unconjugated interferon-α has full interferon activity, but is readily absorbed from the subcutaneous tissue, so little tissue reaction occurs. For the pegylated interferon-α, Pegasys has less activity than PEGIntron (7% vs. 37% of unconjugated interferon-α activity), however the absorption of Pegasys is significantly slower. Absorption half lives of Pegasys vs. PEGIntron are 50 hours and 4.6 hours, respectively, (Foster, Aliment Pharmacol Ther 2004; 20: 825-830), leading to a higher tissue exposure to interferon-α activity, and therefore a higher risk of injection site reaction.

Administration of interferon alpha as a carrier-linked prodrug can reduce the incidence of injection site reactions. As described above, pegylation significantly reduces the activity of interferon. Furthermore, activity of the interferon conjugate is also governed by the attachment site of the PEG molecule. As described by Foser et al. (Foser et al. Protein Expression and Purification 30 (2003) 78-87) pegylation at 9 different lysines of interferon-α2a, led to 9 positional isomers with different activities. The isomers isolated were pegylated at Lys(31), Lys(134), Lys(70), Lys(83), Lys(121), Lys(131), Lys(49), Lys(112), and Lys(164). No pegylation was observed on Lys(23), Lys(133), and the N-terminal PEG, possibly due to steric hinderance at these positions.

Some of the problems relating to permanent PEGylation can be addressed by attaching the PEG molecule or another polymeric carrier to the protein drug via a transient linker resulting in a carrier-linked prodrug. Through this reversible approach, fully active free drug can be released from a prodrug into the blood circulation.

Carrier-linked prodrugs and transient linker systems for such a reversible approach are in general described e.g. in WO-A 2004/089280, WO-A 2005/099768 or U.S. Pat. No. 6,504,005 (see also H. Tsubery et al., J. Biol. Chem. 2004, 279 (37), 38118-38124).

In general, carrier-linked prodrugs require the presence of a cleavable functional group connecting drug and carrier. Functional groups that involve a drug-donated amino group such as aliphatic amide or carbamate bonds are usually very stable against hydrolysis and the rate of cleavage of the amide bond would be too slow for therapeutic utility in a prodrug system. If such stable linkages are to be used in carrier-linked prodrugs, cleavage of the functional group is not possible in a therapeutically useful timeframe without biotransformation. In these cases, the linker may display a structural motif that is recognized as a substrate by a corresponding endogenous enzyme. In such a case, the cleavage of the functional bond involves a complex comprising the enzyme. Examples for such biotransformation-dependent carrier-linked prodrugs employ peptide linkers that are recognized by endogenous proteases and cleaved enzymatically.

Enzyme levels may differ significantly between individuals resulting in biological variation of prodrug activation by the enzymatic cleavage. Enzyme levels may also vary depending on the site of administration. For instance it is known that in the case of subcutaneous injection, certain areas of the body yield more predictable therapeutic effects than others. Such high level of interpatient variability is not desirable. Furthermore, it is difficult to establish an in vivo-in vitro correlation of the pharmacokinetic properties for such enzyme-dependent carrier-linked prodrugs. In the absence of a reliable in vivo-in vitro correlation optimization of a release profile becomes a cumbersome task.

In order to avoid patient-to-patient and injection site variability, it is desirable to employ carrier-linked prodrugs that exhibit cleavage kinetics in a therapeutically useful timeframe without the requirement for additional enzymatic contribution to cleavage. Especially for high molecular weight carriers (polymeric carriers), specifically for branched polymeric carriers, access to the connecting functional group may be restricted for enzymes due to sterical crowding.

Biotransformation-dependent linkers may exhibit different cleavage rates at the site of injection (subcutaneous or intramuscular tissue) and in the blood stream. This is an undesirable characteristic as it compromises in vitro and in vivo correlations and can relate to protracted release, slow-onset of action and poor in vitro-in vivo correlation.

Therefore there exists a need to devise carrier-linked prodrugs that exhibit auto-cleavage.

In order to introduce lability into auto-cleavable groups such as amides or carbamates, it is necessary to engineer structural chemical components into the carrier in order to act for instance as neighbouring groups in proximity to the functional auto-cleavable group. Such auto-cleavage inducing chemical structures that exert control over the cleavability of the prodrug amide bond are termed auto-cleavage inducing groups. Auto-cleavage inducing groups can have a strong effect on the rate of cleavage of a given functional group connecting carrier and biologically active moiety.

A carrier-free system with at least one 2-sulfo-9-fluorenylmethoxycarbonyl (FMS) group and interferon alpha is described in EP-B 1 337 270 (see also Y. Shechter et al., PNAS 2001 98 (3), 1212-1217).

For the delivery of interferon-α2 Peleg-Shulman et al., J. Med. Chem. 2004, 47, 4897-4904, have explored the application of reversible PEGylation by incorporation of a reversible 2-sulfo-9-fluorenylmethoxycarbonyl linker between a 40 kDa PEG and interferon-α2. They demonstrated prolonged release of interferon-α2 with a half life of about 3 days at pH 8.5 and 37° C. The terminal half life of the reversible PEGylated interferon was estimated from the data generated from i.v. injection to be around 30 hours. The hydrolysis of the reversible linker described by this group is strongly biotransformation dependent as it is controlled not only by pH and temperature but also strongly affected by blood plasma nucleophilicity, which means that the interferon release rate will vary between plasma and the subcutaneous tissue. Due to the slow absorption of pegylated interferon conjugate from the subcutaneous tissue, this conjugate is characterized by slow onset of action, as liberation of active interferon occurs primarily in the plasma.

A different approach to sustained release is given by polymer formulation of interferon. This approach has been utilized by companies Biolex and OctoPlus for sustained release of interferon alpha is formulated in poly(ether-ester) microspheres, from which interferon is released continuously as described in WO-A 2006/085747. However the use of such particles as polymeric carrier results in a very low weight ratio of drug to carrier, i.e. the formulation contains much higher amounts of carrier material (e.g. polymer) than drug substance. However, as commonly observed with these types of drugs, such formulation is characterized by burst release, as evident from the pharmacokinetic data presented in L. De Leede et al., Journal of Interferon & Cytokine Research 2008, 28, 113-122, where the pharmacokinetic profile displays two peaks, the first due to initial burst and a second due to release from the formulation. This initial burst will most likely increase the flu-like adverse effects commonly encountered with interferon treatment. Furthermore, as these types of drugs release active drug with full activity from the site of injection for prolonged periods of time, leading to constant tissue exposure interferon-α, it is likely that the patient will suffer injection site reactions similar to those observed with existing treatments.

In order to overcome the problems associated with current technologies, there is a continuing need for new pharmaceutical compositions and prodrugs.

Thus an object of the present invention is to provide such pharmaceutical compositions and prodrugs with advantageous properties relating to release kinetics but preferentially also with regard to drug load, reduced side-effects and injection site reactions, body distribution, viral relapse rate and the like. Accordingly, the present invention provides a pharmaceutical composition comprising a water-soluble polymeric carrier linked prodrug of interferon alpha, wherein the prodrug is capable of releasing free interferon alpha, wherein the release half life under physiological conditions is at least 4 days.

Another aspect of the present invention is a water-soluble polymeric carrier linked prodrug of interferon alpha as defined above.

“Pharmaceutical composition” means one or more active ingredients, and one or more inert ingredients, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a prodrug of the present invention and one or more pharmaceutically acceptable inert ingredients.

The term “inert ingredient” refers to a diluent, adjuvant, excipient, or vehicle with which the therapeutic is administered. Such pharmaceutically acceptable inert ingredients can be sterile liquids, such as water and oils, including those of petroleum, animal, vegetable or synthetic origin, including but not limited to peanut oil, soybean oil, mineral oil, sesame oil and the like. Water is a preferred part of the composition. Saline and aqueous dextrose are preferred ingredients when the pharmaceutical composition is administered intravenously. Saline solutions and aqueous dextrose and glycerol solutions are preferably employed as liquid parts of the composition for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, powders, sustained-release formulations and the like. The composition can be formulated as a suppository, with traditional binders such as triglycerides. Examples of suitable pharmaceutical compositions are described in “Remington's Pharmaceutical Sciences” by E. W. Martin. Such compositions will contain a therapeutically effective amount of the therapeutic, preferably in purified form, together with a suitable amount of other ingredients so as to provide the form for proper administration to the patient. The formulation should suit the mode of administration.

A pharmaceutical composition of the present invention may comprise one or more additional compounds as active ingredients. The active ingredients may be comprised in one or more different pharmaceutical compositions (combination of pharmaceutical compositions). Thus the pharmaceutical composition of the present invention may be useful in mono- or combination therapy using one or more pharmaceutical compositions.

“Dry composition” means that the polymeric carrier-linked interferon alpha prodrug composition is provided in a dry form in a container. Suitable methods for drying are spray-drying and lyophilization (freeze-drying). Such dry composition of polymeric carrier-linked interferon alpha prodrug has a residual water content of a maximum of 10%, preferably less than 5% and more preferably less than 2% (determined according to Karl Fischer). The preferred method of drying is lyophilization. “Lyophilized composition” means that the polymeric carrier-linked interferon alpha prodrug composition was first frozen and subsequently subjected to water reduction by means of reduced pressure. This terminology does not exclude additional drying steps which occur in the manufacturing process prior to filling the composition into the final container.

In a “liquid composition” the polymeric carrier-linked interferon alpha prodrug is provided in such form, that the prodrug is dissolved in a suitable solvent, such as water, optionally containing buffers.

“Lyophilization” (freeze-drying) is a dehydration process, characterized by freezing a composition and then reducing the surrounding pressure and, optionally, adding heat to allow the frozen water in the composition to sublime directly from the solid phase to gas. Typically, the sublimed water is collected by desublimation.

“Reconstitution” means the restoration of the composition's condition prior to drying, such as a solution or suspension, by adding a liquid prior to administrating the composition to a patient in need thereof. The liquid may contain one or more excipients.

“Reconstitution solution” refers to the liquid used to reconstitute the dry composition of a polymeric carrier-linked interferon alpha prodrug prior to administration to a patient in need thereof.

“Container” means any receptacle in which the polymeric carrier-linked interferon alpha prodrug composition is comprised and can be stored in.

“Buffer” or “buffering agent” refers to chemical compounds that maintain the pH in a desired range. Physiologically tolerated buffers are, for example, sodium phosphate, succinate, histidine, bicarbonate, citrate and acetate, sulphate, nitrate, chloride, pyruvate. Antacids such as Mg(OH)₂ or ZnCO₃ may be also used. Buffering capacity may be adjusted to match the conditions most sensitive to pH stability.

“Excipients” refers to compounds administered together with the therapeutic agent, for example, buffering agents, isotonicity modifiers, preservatives, stabilizers, anti-adsorption agents, oxidation protection agents, or other auxiliary agents. However, in some cases, one excipient may have dual or triple functions.

A “lyoprotectant” is a molecule which, when combined with a protein of interest, significantly prevents or reduces chemical and/or physical instability of the protein upon drying in general and especially during lyophilization and subsequent storage. Exemplary lyoprotectants include sugars, such as sucrose or trehalose; amino acids such as monosodium glutamate or histidine; methylamines such as betaine; lyotropic salts such as magnesium sulfate; polyols such as trihydric or higher sugar alcohols, e.g. glycerin, erythritol, glycerol, arabitol, xylitol, sorbitol, and mannitol; ethylene glycol; propylene glycol; polyethylene glycol; pluronics; hydroxyalkyl starches, e.g. hydroxyethyl starch (HES), and combinations thereof.

“Surfactant” refers to wetting agents that lower the surface tension of a liquid.

“Isotonicity modifiers” refer to compounds which minimize pain that can result from cell damage due to osmotic pressure differences at the injection depot.

The term “stabilizers” refers to compounds used to stabilize the hydrogel prodrug. Stabilisation is achieved by strengthening of the protein-stabilising forces, by destabilisation of the denatured state, or by direct binding of excipients to the protein.

“Anti-adsorption agents” refers to mainly ionic or non-ionic surfactants or other proteins or soluble polymers used to coat or adsorb competitively to the inner surface of the composition's container. Chosen concentration and type of excipient depend on the effect to be avoided but typically a monolayer of surfactant is formed at the interface just above the CMC value.

“Oxidation protection agents” refers to antioxidants such as ascorbic acid, ectoine, glutathione, methionine, monothioglycerol, morin, polyethylenimine (PEI), propyl gallate, vitamin E, chelating agents such aus citric acid, EDTA, hexaphosphate, thioglycolic acid.

“Antimicrobial” refers to a chemical substance that kills or inhibits the growth of microorganisms, such as bacteria, fungi, yeasts, protozoans and/or destroys viruses.

“Sealing a container” means that the container is closed in such way that it is airtight, allowing no gas exchange between the outside and the inside and keeping the content sterile.

In a preferred embodiment the pharmaceutical composition is a composition for subcutaneous administration, intramuscular administration or intravenous injection. These are examples of preferred administration routes for treatment of a relevant disorder/disease as described herein.

The pharmaceutical composition of the present invention comprises as active ingredient a water-soluble polymeric carrier linked prodrug of interferon alpha.

The term “prodrug” means in accordance with the definition given by IUPAC any compound that undergoes transformation in vivo before exhibiting its pharmacological effects. Prodrugs can thus be viewed as drugs containing specialized non-toxic protective groups used in vivo in a transient manner to alter or to eliminate undesirable properties in the parent molecule.

The term “carrier linked prodrug” means a prodrug that contains a temporary linkage of a given active substance with a transient carrier group that produces improved physicochemical or pharmacokinetic properties and that can be removed in vivo, usually by a hydrolytic cleavage.

The terms “drug”, “biologically active molecule”, “biologically active moiety”, “biologically active agent”, “active agent”, and the like mean any substance which can affect any physical or biochemical properties of a biological organism, including but not limited to viruses, bacteria, fungi, plants, animals, and humans. In particular, as used herein, biologically active molecules include any substance intended for diagnosis, cure, mitigation, treatment, or prevention of disease in humans or other animals, or to otherwise enhance physical or mental well-being of humans or animals.

A “therapeutically effective amount” of interferon alpha as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician. Within the scope of this invention, therapeutically effective amount relates to dosages that aim to achieve therapeutic effect for an extended period of time, such as for one week or longer, preferably for one to four weeks.

The term “polymeric carrier” according to the present invention means a polymer preferably selected from the group consisting of polyalkoxy polymers (which are preferred, especially polyethylene glycols), hyaluronic acid and derivatives thereof, hydroxyalkyl starch and derivatives thereof, polyvinyl alcohols, polyoxazolines, polyanhydrides, poly(ortho)esters, polycarbonates, polyurethanes, polyacrylic acids, polyacrylamides, polyacrylates, polymethacrylates, polyorganophosphazenes, polysiloxanes, polyvinylpyrrolidone, polycyanoacrylates, polyamides and polyesters and corresponding block copolymers.

The term “interferon alpha” or “interferon α” according to the present invention means a compound belonging to the class of alpha-interferons (IFN-alpha or IFN-α). Alpha-interferons comprise a number of native and modified proteins with similar molecular weight and functionality. Leukocytes are one of the major origins of these proteins in humans. At least 23 different native subtypes and several modified versions of IFN-α are known, some of which are available in pharmaceutical products. The presently most important members of the IFN-α group are the recombinant variants of IFN-α-2a and IFN-α-2b. Another recombinant IFN-α used in therapy is IFNalfacon-1.

The term “free interferon alpha” means the released interferon alpha as defined above after cleavage of the linkage to the carrier in the prodrug of the present invention.

The term “release half-life under physiological conditions” means the time after which 50% of carrier-linked prodrug is hydrolyzed in aqueous buffered solutions containing at least 80% human plasma at pH of around 7.4 (pH 6.8 to pH 7.8) and temperature of about 37° C. (35° C. to 40° C.), preferably pH=7.4 and 37° C.

The term “water-soluble polymeric carrier linked prodrug” means a polymeric carrier linked prodrug that is soluble in buffer at pH 7.4 and 37° C. Typically, a water-soluble prodrug will transmit at least 75%, more preferably at least 95%, of light of a wavelength visible to the human eye transmitted by the same solution after filtering. On a weight basis, a water soluble prodrug at a concentration used for human dosing will preferably be at least about 35% (by weight) soluble in water, still more preferably at least about 50% (by weight), still more preferably at least about 70% (by weight), still more preferably at least about 85% (by weight), still more preferably at least about 95% (by weight) or completely soluble in water.

The release half life of the pharmaceutical composition of the present invention is at least 4 days, preferably at least 5 days, e.g. at least 4 days, 5 days, 6 days, one week, 8 days, 9 days, 10 days, 11 days, 12 days, 13 days, 2 weeks, 3 weeks, 1 month or more up to 100 days. Preferably, the release half life of the pharmaceutical composition of the present invention is at least 96 hours, 120 hours, more preferably at least 180 hours, more preferably at least 240 hours, more preferably at least 300 hours. Also preferably, the release half life of the pharmaceutical composition of the present invention is from 120 to 520; more preferably from 180 hours to 460 hours, more preferably from 240 hours to 400 hours, more preferable 300 hours to 360 hours.

Preferably, the molecular weight of the polymeric carrier is in the range of from 40 kDa to 200 kDa; more preferably, in the range of from 40 kDa to 120 kDa; even more preferably, in the range of 60 kDa to 120 kDa; even more preferably, in the range of from 60 kDa to 100 kDa. Preferably, the polymeric carrier is branched.

Preferably, the interferon alpha is transiently linked to the polymeric carrier such that the release of free interferon alpha is effected through auto-cleavage of an auto-cleavable functional group or linker. Preferably, the auto-cleavable functional group forms together with a primary amino group of interferon alpha a carbamate or amide group.

In such a water-soluble polymeric carrier linked prodrug system, measured activity will have two contributions, one from the released free drug entity and one from the not yet cleaved prodrug. In order to differentiate the activity of the carrier linked prodrug from the released free drug, the term “residual activity” herein is understood as the portion of the measured carrier linked prodrug activity that may be attributed to the prodrug molecule. In order to assess the extent of residual activity, permanent linker conjugates are useful for the investigation of the therapeutic utility of a carrier linked prodrug as they allow for assessment of residual activity if the same carrier is employed in both the prodrug and the permanent linker conjugate.

In the pharmaceutical composition of the present invention interferon alpha is covalently bound to the polymeric carrier. More preferably, primary amino functions of interferon alpha are used. Even more preferably, interferon is conjugated through a lysine side chain or N-terminus. The polymeric carrier can be covalently bound via one or more bonds, like two, three, or four bonds. Preferably, only one or two bonds are present; even more preferably, one bond is present. The polymeric carrier can be formed by two or more polymers, which are bound to interferon alpha, said polymers are not interconnected. In this case the molecular mass of the polymeric carrier is represented by the sum of the molecular masses of the two or more polymers. Preferably, in case the polymeric carrier is formed by two or more polymers it is preferred that only two polymers form the polymeric carrier.

The term “auto-cleavage” herein is therefore understood as rate-limiting cleavage of the bond between a transient linker and the drug molecule interferon alpha in an aqueous buffered solution of pH 7.4 and 37° C. Auto-cleavage does not require the presence of enzyme. This auto-cleavage is controlled by an auto-cleavage inducing group, which is part of the carrier linked prodrug. The auto-cleavage inducing group may be present as such or in a masked form so that unmasking is required before the auto-cleavage mechanism can start. The term “transient linkage” or “transient linker” herein is understood as describing the lability of the linkage between the polymeric carrier and interferon alpha in the prodrug. In such transient linkages, interferon alpha is auto-cleaved from the corresponding prodrug with a release half-life of up to 100 days.

In contrast the term “permanent linker” refers to a carrier linked conjugate with a half-life of hydrolysis of at least 100 days. The term “permanent linker” refers to a polymeric carrier linked conjugate to an interferon alpha-donated primary amino group preferably by formation of an aliphatic amide or aliphatic carbamate. If such permanent linker is used, a resulting polymeric carrier linked conjugate is usually very stable against hydrolysis and the rate of cleavage of the amide or carbamate bond would not allow for therapeutic application as prodrug.

Auto-cleaving polymeric carrier linked prodrugs of the present invention are preferably characterized by exhibiting strong in vitro-in vivo correlation. The in vitro cleavage rate of a carrier-linked prodrug may be obtained by measuring the concentration of free drug in a sample of carrier-linked prodrug in protein-free buffered solution of pH 7.4 at 37° C. over time. For instance, the carrier-linked prodrug may be dissolved in aqueous buffer at pH 7.4 (e.g. 20 mM sodium phosphate, 135 mM NaCl, 3 mM EDTA) and incubated at 37° C. Samples may be taken at time intervals and analyzed by size exclusion chromatography using UV detection at 215 nm on a Superdex 200 column. Peaks corresponding to liberated drug may be integrated and plotted against incubation time. Curve fitting software may be applied to determine a first-order cleavage rate and corresponding in vitro release half-life. Accordingly, the “in vitro release half-life” is the time after which 50% of carrier-linked prodrug are cleaved in protein-free buffer at pH 7.4 at 37° C.

To obtain a correlation of prodrug cleavage rates in vitro and in vivo, it would be desirable to measure the time in which 50% of the initial proportion of interferon alpha is released from the interferon prodrug after administration to the human body. Unfortunately such measurement is not easy to perform, also because the rate of clearance of the carrier-linked prodrug from the blood circulation would have to be taken into account.

It is therefore preferred to determine the carrier-linked prodrug cleavage under physiological conditions. “Physiological conditions” means in vitro or in vivo condition, identical or resembling, the pH and temperature conditions in the human body at the injection site and in the blood stream. More specifically, “physiological conditions” is referring to solutions containing at least 80% human plasma at pH of around 7.4 (pH 6.8 to pH 7.8) and temperature of about 37° C. (35° C. to 40° C.), preferably pH=7.4 and 37° C.

For instance the carrier-linked prodrug may be dissolved in 4/1 (v/v) human plasma/50 mM sodium phosphate buffer at pH 7.4 and filtered through a 0.22 μm filter and incubated at 37° C. Samples may be taken at time intervals and analyzed by an ELISA (e.g. in the case of alpha interferon VeriKine™ Human IFN-Alpha Serum Sample ELISA, PBL Interferonsource, USA, may be employed). Polymeric carrier linked prodrugs of IFN according to the invention would show lower signals in an ELISA as compared to free IFN at the same concentration due to the shielding of the IFN by the conjugated carrier polymer against the antibodies used in the ELISA. Released free IFN may be determined based on the increase of the ELISA signal over time and a calibration curve using unconjugated IFN and amount of liberated free IFN may be plotted against incubation time. Curve fitting software may be applied to determine a first-order cleavage rate and corresponding release half-life.

Correspondingly, the rate of auto-cleavage under physiological conditions can be used to estimate the in vivo cleavage rate of a polymeric carrier linked prodrug and to obtain an in vitro-in vivo correlation. As outlined above it is desirable to obtain an in vitro-in vivo correlation that is as close as possible, i.e. identical or almost identical hydrolysis rates are observed in vitro and under physiological conditions. In order for a polymeric carrier linked prodrug to exhibit self-cleaving characteristics, the in release half-life under physiological conditions may not be less than 50% of the in vitro release half-life.

It is also preferred, that free interferon released from a corresponding polymeric carrier linked prodrug is liberated in an unmodified, traceless fashion, i.e. neither carrier nor linker moieties or fragments or residues thereof remain attached to the interferon after cleavage.

In a preferred embodiment the prodrug of the present invention in the pharmaceutical composition of the present invention is represented by formula (AA)

IFN-NH-L^{a}-S⁰ (AA),

wherein

IFN-NH represents the interferon alpha residue;

L^{a} represents a functional group, which is auto-cleavable by an auto-cleavage-inducing group G^{a};

S⁰ is a branched polymer chain comprising the auto-cleavage inducing group G^{a},

and wherein the molecular weight of the prodrug without the IFN-NH is at least 40 kDa and at most 200 kDa, more preferred at least 40 kDa and at most 120 kDa, more preferred at least 60 kDa and at most 120 kDa; even more preferred at least 60 kDa and at most 100 kDa.

Preferably, S⁰ is a polymer chain having a molecular weight of at least 5 kDa comprising an at least first branching structure BS¹, the at least first branching structure BS¹ comprising an at least second polymer chain S¹ having a molecular weight of at least 4 kDa, wherein the molecular weight of the prodrug without the IFN-NH is at least 40 kDa and at most 200 kDa, more preferred at least 40 kDa and at most 120 kDa, more preferred at least 60 kDa and at most 120 kDa; even more preferred at least 60 kDa and at most 100 kDa, and wherein at least one of S⁰, BS¹, S¹ further comprises the auto-cleavage inducing group G^{a}.

Preferably, the branching structure BS¹ further comprises an at least third polymer chain S² having a molecular weight of at least 4 kDa or at least one of S⁰, S¹ comprises an at least second branching structure BS² comprising the at least third polymer chain S² having a molecular weight of at least 4 kDa, wherein the molecular weight of the prodrug without the IFN-NH is at least 40 kDa and at most 200 kDa, more preferred at least 40 kDa and at most 120 kDa, more preferred at least 60 kDa and at most 120 kDa; even more preferred at least 60 kDa and at most 100 kDa, and wherein at least one of S⁰, BS¹, BS², S¹, S² further comprises the auto-cleavage inducing group G^{a}.

Preferably, at least one of the branching structures BS¹, BS² comprises a further fourth polymer chain S³ having a molecular weight of at least 4 kDa or one of S⁰, S¹, S² comprises a third branching structure BS³ comprising the at least fourth polymer chain S³ having a molecular weight of at least 4 kDa and wherein the molecular weight of the prodrug without the IFN-NH is at least 40 kDa and at most 200 kDa, more preferred at least 40 kDa and at most 120 kDa, more preferred at least 60 kDa and at most 120 kDa; even more preferred at least 60 kDa and at most 100 kDa, and wherein at least one of S⁰, BS¹, BS², BS³, S¹, S², S³ further comprises the auto-cleavage inducing group G^{a}.

The position of the branching position, in the preferred embodiment the first or only branching structure BS¹, within the polymer carrier defines the critical distance. The critical distance is the shortest distance between the attachment site of S⁰ to L^{a} and the branching position (BS¹) measured as connected atoms. The length of the critical distance has an effect on the residual activity. The critical distance is preferably less than 50, more preferred less than 20, and most preferred less than 10.

For prodrugs of the present invention having at least two linkages and carriers, it is preferred that the prodrug is represented by formula (AB))

IFN-(NH-L-S⁰)ₙ (AB),

wherein

n is 2, 3, or 4 (preferably n=2);

IFN(-NH)ₙ represents the interferon alpha residue;

each L is independently a permanent functional group L^{p}; or a functional group L^{a}, which is auto-cleavable by an auto-cleavage inducing group G^{a}; and

each S⁰ is independently a polymer chain having a molecular weight of at least 5 kDa, wherein S⁰ is optionally branched by comprising an at least first branching structure BS¹, the at least first branching structure BS¹ comprising an at least second polymer chain S¹ having a molecular weight of at least 4 kDa, wherein at least one of S⁰, BS¹, S¹ further comprises the auto-cleavage inducing group G^{a} and wherein the molecular weight of the prodrug without the IFN(-NH)ₙ is at least 20 kDa and at most 400 kDa, preferred at least 40 kDa and at most 200 kDa, more preferred at least 60 kDa and at most 120 kDa.

Optionally two, three or more polymer chains are present in the prodrug of the present invention, e.g. 2, 3, 4, 5, 6, 7, or 8. However each further polymer chain has a molecular weight of at least 4 kDa. The total number of polymer chains is limited by the total weight of the prodrug being at most 400 kDa (without IFN(-NH)ₙ), wherein the molecular weight of the prodrug without the IFN-NH is at least 20 kDa and at most 400 kDa, preferred at least 40 kDa and at most 200 kDa, more preferred at least 60 kDa and at most 120 kDa.

Thus a preferred embodiment of the present invention relates to a composition, wherein at least one of the branching structures BS¹, BS² comprises a further fourth polymer chain S³ having a molecular weight of at least 4 kDa or one of S⁰, S¹, S² comprises a third branching structure BS³ comprising the at least fourth polymer chain S³ having a molecular weight of at least 4 kDa, wherein the molecular weight of the prodrug without the IFN(-NH)ₙ is at least 20 kDa and at most 400 kDa, preferred at least 40 kDa and at most 200 kDa, more preferred at least 60 kDa and at most 120 kDa.

The auto-cleavage inducing group G^{a}, which is necessary for the auto-cleavage of L^{a} is comprised by one of the branching structures or polymer chains. Optionally, one of the branching structures serves as group G^{a} so that the branching structure consists of G^{a} (instead of comprising said group), which is also encompassed by the term “comprising”.

The preparation of a prodrug (AA) typically results in a mixture of prodrugs, where several primary amino groups of IFN are linked to carriers resulting in different mono-linked, different bi-linked, different tri-linked, etc., prodrugs. Corresponding mono-linked, bis-linked or tris-linked prodrugs can be separated by standard methods known in the art, like column chromatography and the like.

In mono-linked carrier prodrugs, the two more polymer chains S⁰, S¹, S², S³ contain a “polymer moiety”, which is characterized by one or more repeating units, which may be randomly, block wise or alternating distributed. In addition, the two or more polymer chains S⁰, S¹, S², S³ show an end group, which is typically a hydrogen atom or an alkyl group having from 1 to 6 carbon atoms, which may be branched or unbranched, e.g. a methyl group, especially for poly(ethylene)glycol (PEG) based polymer chains resulting in so called mPEGs.

It is pointed out that the polymer moieties within the two or more polymer chains S⁰, S¹, S², S³ may have further chain-like substituents, originating from the repeating units and resulting in chains having less than 4 kDa of molecular weight and which are not considered as polymer chains S⁰, S¹, S², S³ etc. Preferably, the two or more polymer chains S⁰, S¹, S², S³ carry substituents of less than 4 kDa molecular weight.

The two or more polymer chains S⁰, S¹ and S², S³ typically each contain an interconnecting moiety. G^{a} is present in at least one of the interconnecting moieties. For polymer chains other than S⁰, the interconnecting moiety is the structural element connecting the polymer moiety of for instance S¹ with BS¹ and the polymer moiety of S² with BS². For S⁰, the interconnecting moiety is the structural element connecting L^{a} and BS¹.

Interconnecting moieties may consist of a C₁₋₅₀ alkyl chain, which is branched or unbranched and which is optionally interrupted or terminated by hetero atoms or functional groups selected from the group consisting of —O—; —S—; N(R); C(O); C(O)N(R); N(R)C(O); one or more carbocycles or heterocycles, wherein R is hydrogen or a C₁₋₂₀ alkyl chain, which is optionally interrupted or terminated by one or more of the abovementioned atoms or groups, which further have a hydrogen as terminal atom; and wherein a carbocycle is phenyl; naphthyl; indenyl; indanyl; tetralinyl; C₃₋₁₀ cycloalkyl; and wherein the heterocycle is a 4 to 7 membered heterocyclyl; or 9 to 11 membered heterobicyclyl.

“C₃₋₁₀ cycloalkyl” or “C₃₋₁₀ cycloalkyl ring” means a cyclic alkyl chain having 3 to 10 carbon atoms, which may have carbon-carbon double bonds being at least partially saturated, e.g. cyclopropyl, cyclobutyl, cyclopentyl, cyclohexyl, cyclohexenyl, cycloheptyl, cyclooctyl, cyclononyl, cyclodecyl. Each hydrogen of a cycloalkyl carbon may be replaced by a substituent. The term “C₃₋₁₀ cycloalkyl” or “C₃₋₁₀cycloalkyl ring” also includes bridged bicycles like norbonane or norbonene.

“4 to 7 membered heterocyclyl” or “4 to 7 membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 4 to 7 membered heterocycles are azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine.

“9 to 11 membered heterobicyclyl” or “9 to 11 membered heterobicycle” means a heterocyclic system of two rings with 9 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 9 to 11 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine or pteridine. The term 9 to 11 membered heterobicycle also includes spiro structures of two rings like 1,4-dioxa-8-azaspiro[4.5]decane or bridged heterocycles like 8-aza-bicyclo[3.2.1]octane.

The carbocycle, heterocycle and heterobicycle may be substituted by C₁₋₂₀ alkyl, optionally interrupted or terminated by hetero atoms or functional groups selected from the group consisting of —O—; —S—; N(R); C(O); C(O)N(R); N(R)C(O), wherein R is hydrogen or a C₁₋₁₀ alkyl chain, which is optionally interrupted or terminated by one or more of the abovementioned atoms or groups, which further have a hydrogen as terminal atom.

The polymer moiety of the two or three or more chains S⁰, S¹, S² form the majority part of the chains, preferably at least 90% of the molecular weight of each chain, more preferred at least 95%, even more preferred at least 97.5%. Thus, the basis of the chains is represented by the polymer moiety.

Preferably, the two or more chains S⁰, S¹, S² are independently based on a polymer selected from the group consisting of polyalkoxy polymers (which is preferred, especially poly(ethylene)glycols), hyaluronic acid and derivatives thereof, hydroxyalkyl starch and derivatives thereof polyvinyl alcohols, polyoxazolines, polyanhydrides, poly(ortho esters), polycarbonates, polyurethanes, polyacrylic acids, polyacrylamides, polyacrylates, polymethacrylates, polyorganophosphazenes, polysiloxanes, polyvinylpyrrolidone, polycyanoacrylates, polyamides and polyesters, and corresponding block copolymers.

Preferably, the two or more chains S⁰, S¹, S² are based on the same polymer. Preferably, the two or more chains S⁰, S¹, S² are based on polyalkyoxy polymers. Even more preferred the two or more chains S⁰, S¹, S² are polyethylene glycol based.

The same applies for further chains S³, S⁴, S⁵, etc, accordingly (if present).

The chain S⁰ comprises a branching structure BS¹, so that S¹ is linked to S⁰. For the linkage of S² the branching structure BS¹ may be used or a further branching structure BS² is present, which may be a part of S⁰ or S¹. Accordingly, further branching structures may be present, when further chains are present. For example in case a chain S³ is present it may be linked to BS¹, BS² or a branching structure BS³. The branching structure BS³, if present, may be part of S⁰, S¹, or S².

In general any chemical entity, which allows the branching of a chain, may be used. Preferably, the branching structures are independently selected from the group consisting of at least 3-fold substituted carbocycle, at least 3-fold substituted heterocycle, a tertiary carbon atom, a quaternary carbon atom, and a tertiary nitrogen atom, wherein the terms carbocycle and heterocycle are defined as indicated above.

In publications in the art auto-cleavage inducing groups are sometimes called linkers to discriminate their structure from the carrier. Nevertheless it is often difficult to clearly separate these structural features. Therefore, within the meaning of the present invention the cleavage inducing group G^{a} is considered to be part of the carrier S, comprising at least S⁰, S¹, BS¹. Variation of the chemical nature of G^{a} allows the engineering of the properties of the auto-cleaving properties of a corresponding prodrug to a great extent.

Suitable transient linker structures exhibiting release profiles of interest are described in WO-A 2005/099768. Other transient linker structures are generically/broadly described in e.g. WO-A 2005/034909, WO-A 2005/099768, WO-A 2006/003014 and WO-A 2006/136586.

More transient linker structures are broadly described in e.g. WO-A 99/30727.

Especially, suitable transient linker structures, which are auto-cleavable can be chosen for incorporation into S⁰. The herein selected linker structures are described in detail below.

Ideally, a prodrug of the invention will possess one or more of the following features and/or advantages over current interferon alpha conjugates or formulations; the prodrug can easily be synthesized in good yields, can be purified to provide homogeneous compositions, exhibit activity after auto-cleavage such as in vitro and in vivo and have pharmacodynamic effects superior to unmodified interferon alpha and previously described conjugates.

Auto-cleavage inducing chemical structures that exert control over the cleavability of the prodrug bond are termed auto-cleavage inducing groups (G^{a} according to the definition of L^{a} in formula (AA)). Auto-cleavage inducing groups can have a strong effect on the rate of cleavage of a given functional group L^{a}.

Preferred L^{a} is selected from the group consisting of C(O)—O—, and C(O)—, which forms together with a primary amino group of interferon alpha a carbamate or amide group.

Thus, a composition of the present invention is preferred, wherein L^{a} is selected from the group consisting of C(O)—O—, and C(O)—, which forms together with the primary amino group of IFN a carbamate or amide group resulting in formula (AA1) or (AA2)

IFN-NH—C(O)O—S⁰ (AA1),

IFN-NH—C(O)—S⁰ (AA2).

The following sections will list various structural components that may function as auto-cleavage inducing groups G^{a}.

The group G^{a} represents an auto-cleavage inducing group. G^{a} may be present as such or as a cascade auto-cleavage inducing group, which is unmasked to become effective by means of an additional hydrolytic or enzymatic cleavage step. If G^{a} is present as such, it governs the rate-limiting cleavage of L^{a}.

Preferably, transformation of G^{a} may induce a molecular rearrangement within S⁰ such as a 1,4- or 1,6-elimination. The rearrangement renders L^{a} so much more labile that its cleavage is induced. The transformation of G^{a} is the rate-limiting step in the cascade mechanism. Ideally, the cleavage rate of the transient linkage is identical to the desired release rate for the drug molecule in a given therapeutic scenario. In such a cascade system based on elimination, it is desirable that the cleavage of L^{a} is substantially instantaneous after its lability has been induced by transformation of G^{a}. In addition it is desirable that the rate-limiting cleavage kinetics proceed in a therapeutically useful timeframe without the requirement for additional enzymatic contribution in order to avoid the drawbacks associated with predominantly enzymatic cleavage discussed above.

R. B. Greenwald, A. Pendri, C. D. Conover, H. Zhao, Y. H. Choe, A. Martinez, K. Shum, S. Guan, J. Med. Chem., 1999, 42, 3657-3667 & PCT Patent Application WO-A 99/30727 described a methodology for synthesizing poly(ethylene glycol) prodrugs of amino-containing small molecule compounds based on 1,4- or 1,6-benzyl elimination. In this approach the amino group of the drug molecule is linked via a carbamate group to a PEGylated benzyl moiety. The poly(ethylene glycol) is attached to the benzyl group by ester, carbonate, carbamate, or amide bonds. The release of PEG from the drug molecule occurs through a combination of autohydrolysis and enzymatic cleavage. The cleavage of the release-triggering masking group is followed in this approach by the classical and rapid 1,4- or 1,6-benzyl elimination. This linker system was also used for releasable poly(ethylene glycol) conjugates of proteins (S. Lee, R. B. Greenwald et al. Bioconj. Chem. 2001, 12 (2), 163-169). Lysozyme was used as model protein because it loses its activity when PEGylation takes place on the epsilon-amino group of lysine residues. Various amounts of PEG linker were conjugated to the protein. Regeneration of free protein from the PEG conjugates occurred in rat plasma or in non-physiological high pH buffer. See also F. M. H. DeGroot et al. (WO-A 2002/083180 and WO-A 2004/043493), and D. Shabat et al. (WO-A 2004/019993).

Thus, L^{a} is a carbamate functional group, the cleavage of said group is induced by a hydroxyl or amino group of G^{a} via 1,4- or 1,6 benzyl elimination of S⁰, wherein G^{a} contains ester, carbonate, carbamate, or amide bonds that undergo rate-limiting transformation. In effect, G^{a} may be cleaved off by hydrolysis.

Accordingly, a composition of the present invention is preferred, wherein L^{a} forms together with the amino group of interferon alpha a carbamate functional group, the cleavage of said group is induced by a hydroxyl or amino group of G^{a} via 1,4- or 1,6 benzyl elimination of S⁰, wherein G^{a} contains ester, carbonate, carbamate, or amide bonds that undergo rate-limiting transformation.

G^{a} may contain a cascade cleavage system that is enabled by components of G^{a} that are composed of a structural combination representing the aforementioned precursor. A precursor of G^{a} may contain additional transient linkages such as an amide, ester or a carbamate. The stability or susceptibility to hydrolysis of the precursor's temporary linkage (e.g. carbamate) may be governed by autohydrolytic properties or may require the activity of an enzyme.

More specifically, preferred groups L^{a} and G^{a} with specific spacer moieties for S⁰ are described below. A preferred structure according to WO-A 2005/099768 is selected from the general formula (I) and (II):

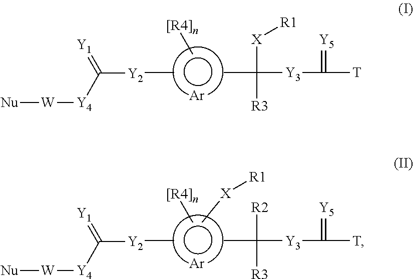

wherein T represents IFN-NH; X represents a spacer moiety; Y₁ and Y₂ each independently represent O, S or NR₆; Y₃ represents O or S; Y₄ represents O, NR₆ or —C(R₇)(R₈); R₃ represents a moiety selected from the group consisting of hydrogen, substituted or unsubstituted linear, branched or cyclical alkyl or heteroalkyl groups, aryls, substituted aryls, substituted or unsubstituted heteroaryls, cyano groups, nitro groups, halogens, carboxy groups, carboxyalkyl groups, alkylcarbonyl groups or carboxamidoalkyl groups; R₄ represents a moiety selected from the group consisting of hydrogen, substituted or unsubstituted linear, branched or cyclical alkyls or heteroalkyls, aryls, substituted aryls, substituted or unsubstituted heteroaryl, substituted or unsubstituted linear, branched or cyclical alkoxys, substituted or unsubstituted linear, branched or cyclical heteroalkyloxys, aryloxys or heteroaryloxys, cyano groups and halogens; R₇ and R₈ are each independently selected from the group consisting of hydrogen, substituted or unsubstituted linear, branched or cyclical alkyls or heteroalkyls, aryls, substituted aryls, substituted or unsubstituted heteroaryls, carboxyalkyl groups, alkylcarbonyl groups, carboxamidoalkyl groups, cyano groups, and halogens; R₆ represents a group selected from hydrogen, substituted or unsubstituted linear, branched or cyclical alkyls or heteroalkyls, aryls, substituted aryls and substituted or unsubstituted heteroaryls; R₁ represents the rest of S⁰ ; W represents a group selected from substituted or unsubstituted linear, branched or cyclical alkyls, aryls, substituted aryls, substituted or unsubstituted linear, branched or cyclical heteroalkyls, substituted or unsubstituted heteroaryls; Nu represents a nucleophile; n represents zero or a positive imager; and Ar represents a multi-substituted aromatic hydrocarbon or multi-substituted aromatic heterocycle.

Within the meaning of the present invention, the group L^{a} is represented by Y₃—C(Y₅)NH— (together with the amino group of IFN), G^{a} is represented by Nu-W—Y₄—C(Y₁)Y₂ and Ar(R₄)ₙ—C(R₃)XR₁ represents S⁰, which preferably further includes at least BS¹ and S¹.

In an alternative embodiment S¹ is attached via Ar or represents R₃. Then the carbon atom adjacent to Y₃ substituted with XR¹ represents the branching structure BS¹, S¹ is terminated with Ar comprising G^{a}. it is evident that in this embodiment terms S⁰ and S¹ are interchangeable.

Preferably, in formula (AA) or (AA1) S⁰ is of formula (AAA1)

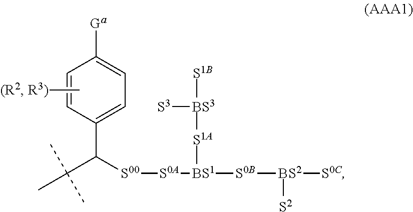

wherein

G^{a} has the meaning as indicated above;

S⁰⁰ is CH₂; or C(O);

S^{0A} is an alkylene chain having less than 50, more preferred less than 20, and most preferred less than 10 carbon atoms, which is optionally interrupted or terminated by one or more groups, cycles or heteroatoms selected from the group consisting of optionally substituted heterocycle; O; S; C(O); and NH;

BS¹, BS², BS³ are independently selected from the group consisting of N; and CH.

S^{0B}, S^{1A} are independently an alkylene chain having from 1 to 25 carbon atoms, which is optionally interrupted or terminated by one or more groups, cycles or heteroatoms selected from the group consisting of optionally substituted heterocycle; O; S; C(O); and NH;

S^{0C}, S^{1B}, are (C(O))ₙ₂(CH₂)ₙ₁(OCH₂CH₂)ₙOCH₃, wherein each n is independently an integer from 90 to 2500, each n1 is independently an integer from 1 to 25 and n2 is 0; or 1

S², S³ are independently hydrogen; or (C(O))ₙ₂(CH₂)ₙ₁(OCH₂CH₂)ₙOCH₃, wherein each n is independently an integer from 90 to 2500, each n1 is independently an integer from 1 to 25 , and n2 is 0; or 1;

R², R³ are independently selected from the group consisting of hydrogen; methyl; ethyl; propyl; isopropyl; butyl; isobutyl; and tert-butyl.

In contrast to the general meaning of the terms S², S³ according to the present invention S², S³ in formula (AAA1) can be hydrogen. Accordingly, none of S², S³ can be hydrogen (resulting in a two fold branched carrier) or one of S², S³ can be hydrogen (resulting in a three fold branched carrier) or both can be hydrogen (resulting in a four fold branched carrier). Thus specifically for the definition of S², S³ in formula (AAA1) these terms do not necessarily represent polymer chains. Accordingly, BS² and BS³ do not necessarily represent branching position.

The term heterocycle means an heterocycle as defined above. Optional substituents are, e.g. oxo (═O), where the ring is at least partially saturated, a branched or unbranched alkyl chain having from one to 6 carbon atoms, or halogen. A preferred substituted heterocycle is succinimide

Preferably, G^{a} in formula (AAA1) is OC(O)—R and R is the partial structure of formula (I) as shown below, wherein R1, R4, R5 and n are defined as given below.

Accordingly, it is preferred that G^{a} is OC(O)—R and R is the partial structure of formula (I)

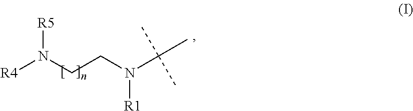

wherein R1, R4, R5 are independently selected from the group consisting of hydrogen; methyl; ethyl; propyl; isopropyl; butyl; isobutyl; and tert.-butyl, and wherein n is 1 or 2.

Even more preferred general aromatic structures are listed below.

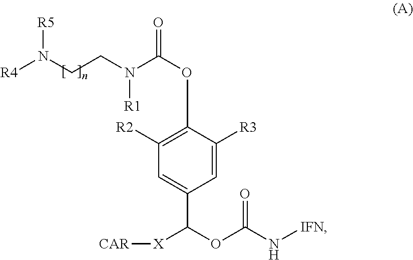

wherein

NH-IFN represents the interferon alpha residue attached to the transient linker;

R1, R2, R3, R4, and R5 are selected independently from hydrogen, methyl, ethyl, propyl, isopropyl, butyl, isobutyl, tertiary butyl,

CAR represents the polymeric carrier residue attached to the transient linker,

n=1 or 2, and

X is selected from C1 to C8 alkyl or C1 to C12 heteroalkyl.

The term “C1 to C12 heteroalkyl” means an alkyl chain having 1 to 12 carbon atoms which are optionally interrupted by heteroatoms, functional groups, carbocycles or heterocycles as defined above.

In a preferred embodiment, in formula (A) L^{a} is represented by the carbamate group attached to interferon alpha, G^{a} is represented by the aromatic oxygen group, the carbonyl attached to it, and the substituent attached to the carbonyl as shown in formula I.

More preferred structures are given by general formula I, which are part of the structure (A) within the general aromatic linker structure above:

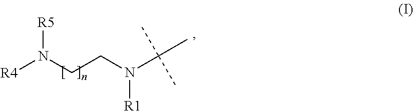

where preferred examples of formula (I) comprise:

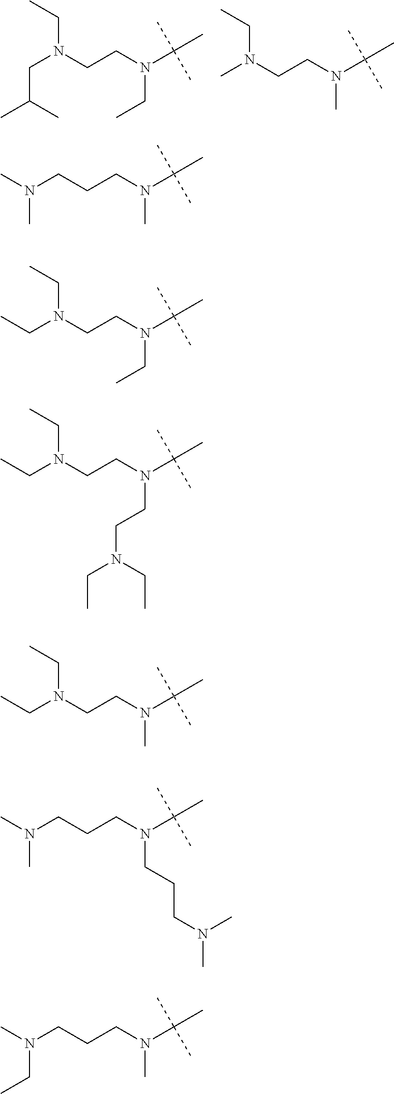

More preferred aromatic structures of formula (II), which are part of the structure (A) within the general aromatic linker structure above:

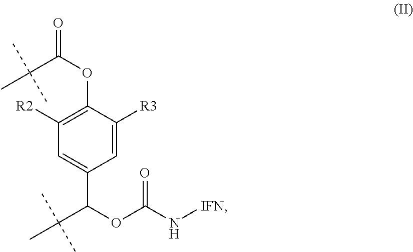

wherein preferred examples of formula (II) comprise:

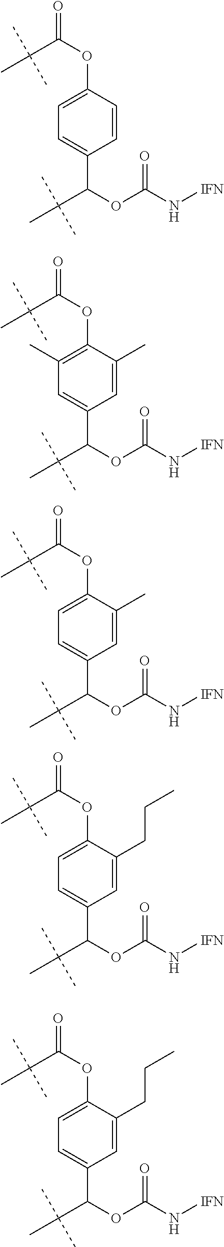

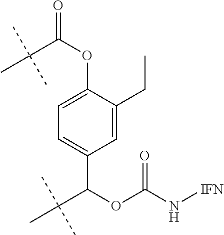

Another preferred embodiment is described in WO-A 2006/136586. Accordingly, the following structures are preferred:

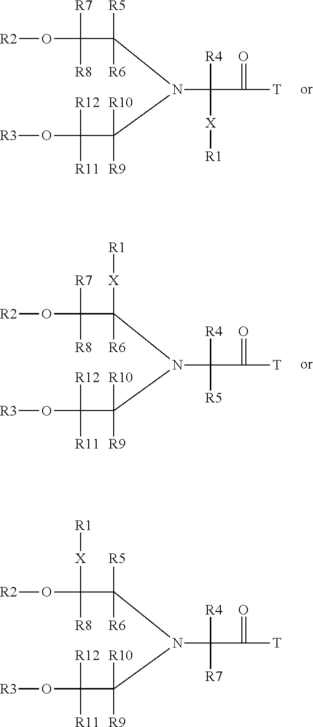

wherein T is NH-IFN;

X is a spacer moiety such as R13-Y1;

Y1 is O, S, NR6, succinimide, maleimide, unsaturated carbon-carbon bonds or any heteratom containing a free electron pair or is absent;

R13 is selected from substituted or non-substituted linear, branched or cyclical alkyl or heteroalkyl, aryls, substituted aryls, substituted or non-substituted heteroaryls;

R2 and R3 are selected independently from hydrogen, acyl groups, or protecting groups for hydroxyl groups;

R4 to R12 are selected independently from hydrogen, X—R1, substituted or non-substituted linear, branched or cyclical alkyl or heteroalkyl, aryls, substituted aryls, substituted or non-substituted heteroaryls, cyano, nitro, halogen, carboxy, carboxamide;

R1 is the rest of S⁰, comprising at least S¹ and BS¹.

In this embodiment L^{a} is an amide group, and G^{a} encompasses the N-branched structure carrying OR2/OR3.

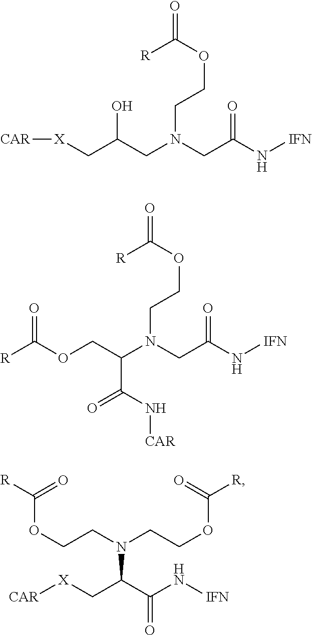

wherein R is selected from hydrogen, methyl, ethyl, propyl and butyl; X is selected from C1 to C8 alkyl or C1 to C12 heteroalkyl and CAR is the polymeric carrier residue.

Also in the preferred and more preferred embodiments CAR means preferably the rest of S⁰, comprising at least S¹, BS¹.

In yet another preferred embodiment, a preferred structure is given by a carrier-linked prodrug D-L, wherein

-D is NH-IFN; and

-L is a

non-biologically active linker moiety -L¹ represented by formula (I),

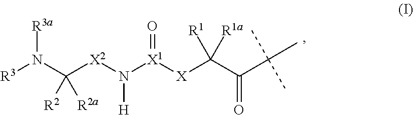

wherein the dashed line indicates the attachment to the amino group of IFN by forming an amide bond;

X is C(R⁴R⁴a); N(R⁴); O; C(R⁴R^{4a})—C(R⁵R⁵a); C(R⁵R^{5a})—C(R⁴R^{4a}); C(R⁴R^{4a})—N(R⁶); N(R⁶)—C(R⁴R^{4a}); C(R⁴R^{4a})—O; or O—C(R⁴R^{4a});

X¹ is C; or S(O);

X² is C(R⁷, R^{7a}); or C(R⁷, R^{7a})—C(R⁸, R^{8a});

X³ is O; S; or N—CN;

R¹, R^{1a}, R², R^{2a}, R³, R^{3a}, R⁴, R^{4a}, R⁵, R5a, R⁶, R⁷, R^{7a}, R⁸, R^{8a} are independently selected from the group consisting of H; and C₁₋₄ alkyl;

Optionally, one or more of the pairs R^{1a}/R^{4a}, R^{1a}/R^{5a}, R^{4a}/R^{5a}, R^{7a}/R^{8a} form a chemical bond;

Optionally, one or more of the pairs R¹/R^{1a}, R²/R^{2a}, R⁴/R^{4a}, R⁵/R^{5a}, R⁷/R^{7a}, R⁸/R^{8a} are joined together with the atom to which they are attached to form a C₃₋₇ cycloalkyl; or 4 to 7 membered heterocyclyl;

Optionally, one or more of the pairs R¹/R⁴, R¹/R⁵, R¹/R⁶, R⁴/R⁵, R⁴/R⁶, R⁷/R⁸, R²/R³ are joined together with the atoms to which they are attached to form a ring A;

Optionally, R³/R^{3a} are joined together with the nitrogen atom to which they are attached to form a 4 to 7 membered heterocycle;

A is selected from the group consisting of phenyl; naphthyl; indenyl; indanyl; tetralinyl; C₃₋₁₀ cycloalkyl; 4 to 7 membered heterocyclyl; and 9 to 11 membered heterobicyclyl; and

wherein L¹ is substituted with one group L²-Z and optionally further substituted, provided that the hydrogen marked with the asterisk in formula (I) is not replaced by a substituent; wherein

L² is a single chemical bond or a spacer; and

Z is the rest of S⁰, comprising at least S¹, BS¹.

In this embodiment L^{a} is represented by an amide group and G^{a} is represented by N(H*)X¹(O) and the chain connecting to N including subtituents of N.

Prodrugs of this type are described in European Patent application N° 08150973.9

Accordingly, a composition of the present invention is preferred, wherein L^{a}-S⁰ is represented by formula (AAA2),

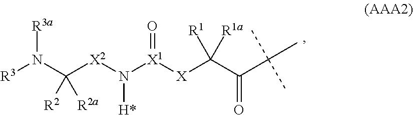

wherein the dashed line indicates the attachment to the primary amino group of IFN so that L^{a} and the amino group form an amide bond;

X is C(R⁴R^{4a}); N(R⁴); O; C(R⁴R^{4a})—C(R⁵R^{5a}); C(R⁵R^{5a})—C(R⁴R^{4a}); C(R⁴R^{4a})—N(R⁶); N(R⁶)—C(R⁴R^{4a}); C(R⁴R^{4a})—O; or O—C(R⁴R^{4a});

X¹ is C; or S(O);

X² is C(R⁷, R^{7a}); or C(R⁷, R^{7a})—C(R⁸, R^{8a});

X³ is O; S; or N—CN;

R¹, R^{1a}, R², R^{2a}, R³, R^{3a}, R⁴, R^{4a}, R⁵, R^{5a}, R⁶, R⁷, R^{7a}, R⁸, R^{8a} are independently selected from the group consisting of H; and C₁₋₄ alkyl;

Optionally, one or more of the pairs R^{1a}/R^{4a}, R^{1a}R^{5a}, R^{4a}/R^{5a}, R^{7a}/R^{8a} form a chemical bond;

Optionally, one or more of the pairs R¹/R^{1a}, R²/R^{2a}, R⁴/R^{4a}, R⁵/R^{5a}, R⁷/R^{7a}, R⁸/R^{8a} are joined together with the atom to which they are attached to form a C₃₋₇ cycloalkyl; or 4 to 7 membered heterocyclyl;

Optionally, one or more of the pairs R¹/R⁴, R¹/R⁵, R¹/R⁶, R⁴/R⁵, R⁴/R⁶, R⁷/R⁸, R²/R³ are joined together with the atoms to which they are attached to form a ring A;

Optionally, R³/R^{3a} are joined together with the nitrogen atom to which they are attached to form a 4 to 7 membered heterocycle;

A is selected from the group consisting of phenyl; naphthyl; indenyl; indanyl; tetralinyl; C₃₋₁₀ cycloalkyl; 4 to 7 membered heterocyclyl; and 9 to 11 membered heterobicyclyl; and

wherein S⁰ is substituted with one group L²-Z and optionally further substituted, provided that the hydrogen marked with the asterisk in formula (I) is not replaced by a substituent; wherein

L² is a single chemical bond or a spacer; and

Z is of formula (AAA2a)

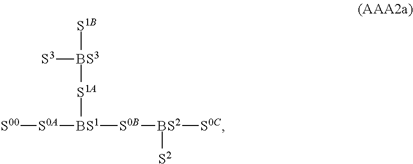

wherein S⁰⁰, S^{0A}, S^{0B}, S^{0C}, S^{1A}, S^{1B}, S², S³, BS¹, BS², and BS³ have the meaning as indicated for formula (AAA1) above.

“Alkyl” means a straight-chain or branched carbon chain. Each hydrogen of an alkyl carbon may be replaced by a substituent.

“C₁₋₄ alkyl” means an alkyl chain having 1-4 carbon atoms, e.g. if present at the end of a molecule: methyl, ethyl, n-propyl, isopropyl, n-butyl, isobutyl, sec-butyl tert-butyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₄ alkyl carbon may be replaced by a substituent.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl; tert-butyl, n-pentyl, n-hexyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₆ alkyl carbon may be replaced by a substituent.

Accordingly, “C₁₋₁₈ alkyl” means an alkyl chain having 1 to 18 carbon atoms and “C₈₋₁₈ alkyl” means an alkyl chain having 8 to 18 carbon atoms. Accordingly, “C₁₋₅₀ alkyl” means an alkyl chain having 1 to 50 carbon atoms.

“C₂₋₅₀ alkenyl” means a branched or unbranched alkenyl chain having 2 to 50 carbon atoms, e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. Each hydrogen of a C₂₋₅₀ alkenyl carbon may be replaced by a substituent as further specified. Accordingly, the term “alkenyl” relates to a carbon chain with at least one carbon carbon double bond. Optionally, one or more triple bonds may occur.

“C₂₋₅₀ alkynyl” means a branched or unbranched alkynyl chain having 2 to 50 carbon atoms, e.g. if present at the end of a molecule: —C≡CH, —CH₂—C≡CH, CH₂—CH₂—C≡CH, CH₂—C≡C—CH₃, or e.g. —C≡C— when two moieties of a molecule are linked by the alkynyl group. Each hydrogen of a C₂₋₅₀ alkynyl carbon may be replaced by a substituent as further specified. Accordingly, the term “alkynyl” relates to a carbon chaim with at lest one carbon carbon triple bond. Optionally, one or more double bonds may occur.

“C₃₋₇ cycloalkyl” or “C₃₋₇ cycloalkyl ring” means a cyclic alkyl chain having 3 to 7 carbon atoms, which may have carbon-carbon double bonds being at least partially saturated, e.g. cyclopropyl, cyclobutyl, cyclopentyl, cyclohexyl, cyclohexenyl, cycloheptyl. Each hydrogen of a cycloalkyl carbon may be replaced by a substituent. The term “C₃₋₇ cycloalkyl” or “C₃₋₇ cycloalkyl ring” also includes bridged bicycles like norbonane or norbonene. Accordingly, “C₃₋₅ cycloalkyl” means a cycloalkyl having 3 to 5 carbon atoms.

Accordingly, “C₃₋₁₀ cycloalkyl” means a cyclic alkyl having 3 to 10 carbon atoms, e.g. C₃₋₇ cycloalkyl; cyclopropyl, cyclobutyl, cyclopentyl, cyclohexyl, cyclohexenyl, cycloheptyl, cyclooctyl, cyclononyl, cyclodecyl. The term “C₃₋₁₀ cycloalkyl” also includes at least partially saturated carbomono- and -bicycles.

“Halogen” means fluoro, chloro, bromo or iodo. It is generally preferred that halogen is fluoro or chloro.

“4 to 7 membered heterocyclyl” or “4 to 7 membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 4 to 7 membered heterocycles are azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine.

“9 to 11 membered heterobicyclyl” or “9 to 11 membered heterobicycle” means a heterocyclic system of two rings with 9 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 9 to 11 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine or pteridine. The term 9 to 11 membered heterobicycle also includes spiro structures of two rings like 1,4-dioxa-8-azaspiro[4.5]decane or bridged heterocycles like 8-aza-bicyclo[3.2.1]octane.

Preferably, X³ is O. Preferably, X is N(R⁴), X¹ is C and X³ is O. Preferably, X² is C(R⁷R^{7a}).

Preferably, L^{a}-S⁰ is selected from the group consisting of

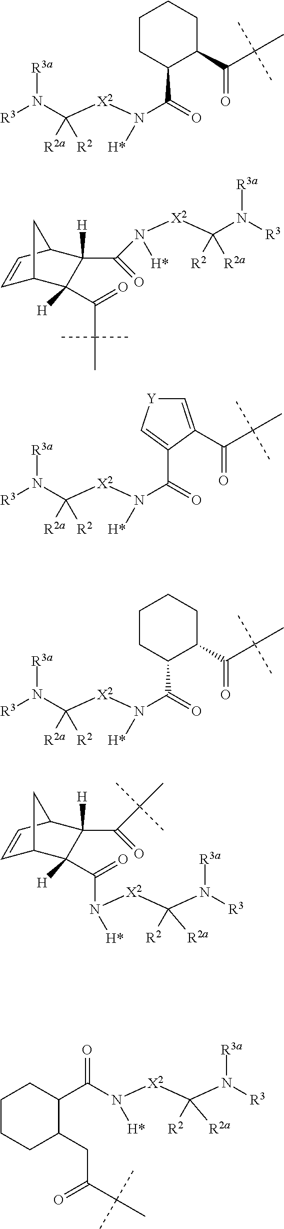

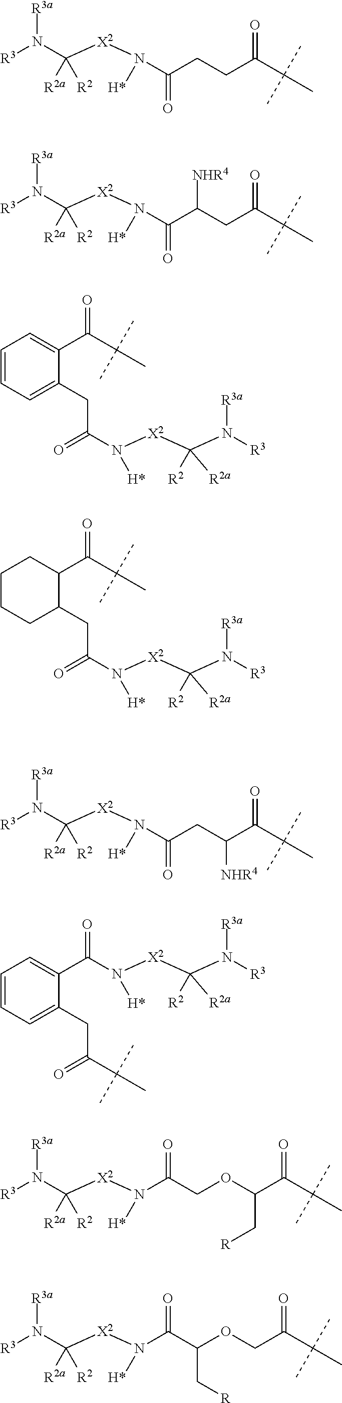

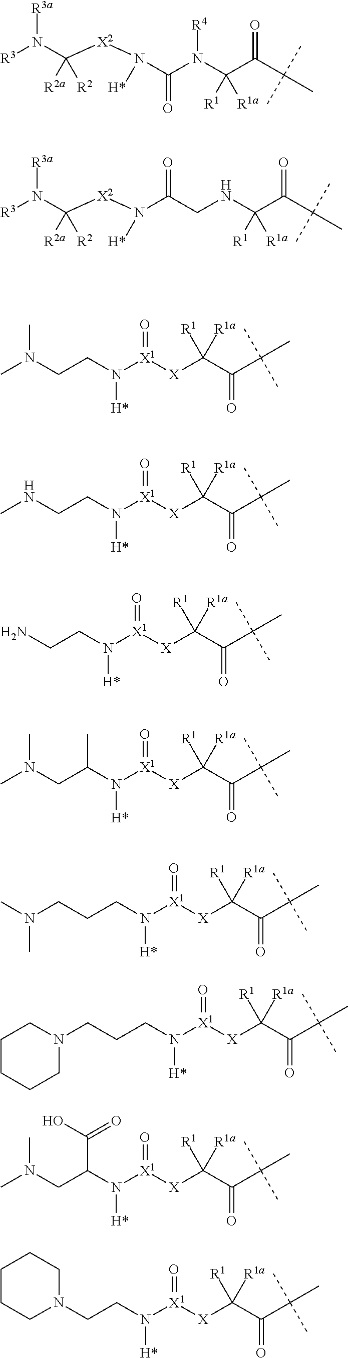

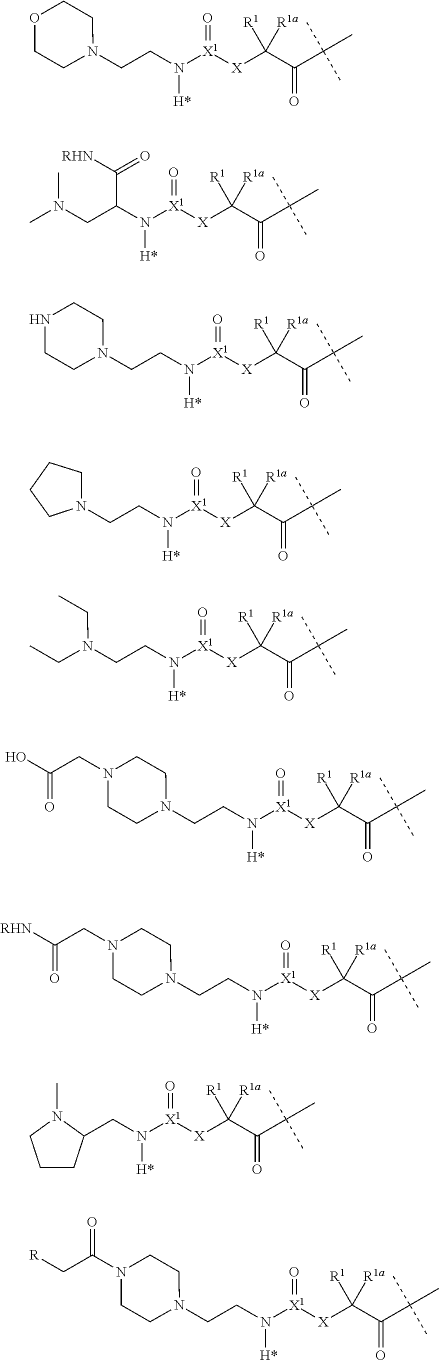

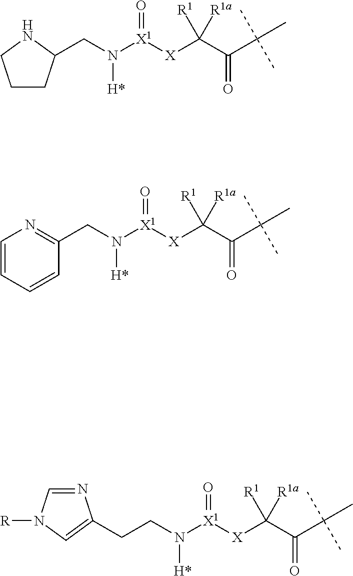

wherein R is H; or C₁₋₄ alkyl; Y is NH; O; or S; and R¹, R^{1a}, R², R^{2a}, R³, R^{3a}, R⁴, X, X¹, X² have the meaning as indicated above.

Even more preferred, L^{a}—S⁰ is selected from the group consisting of

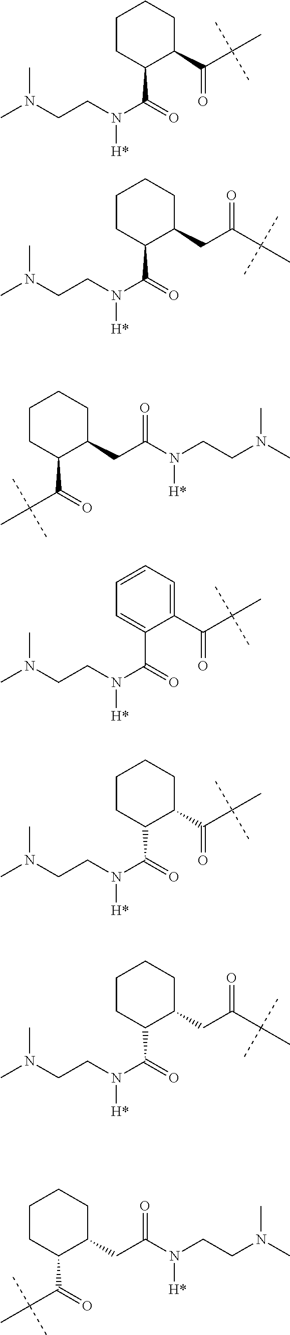

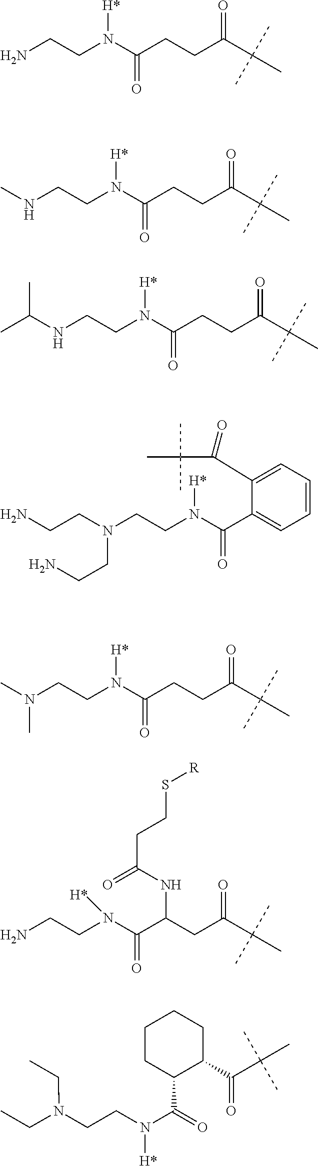

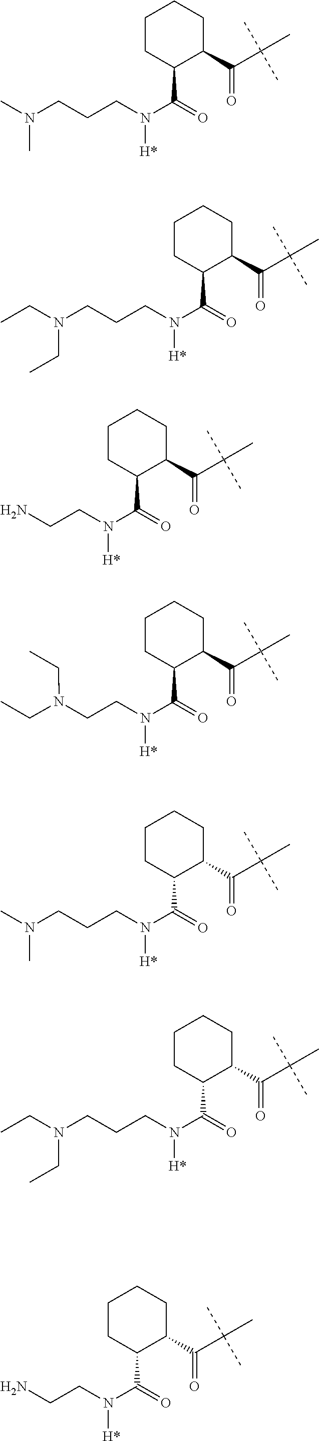

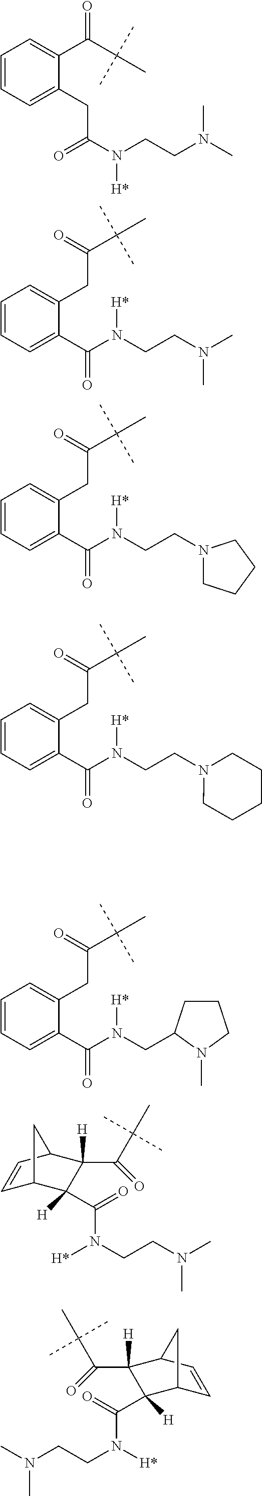

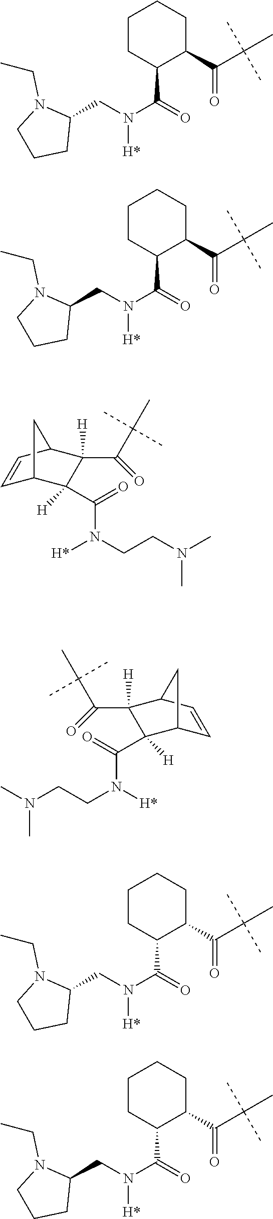

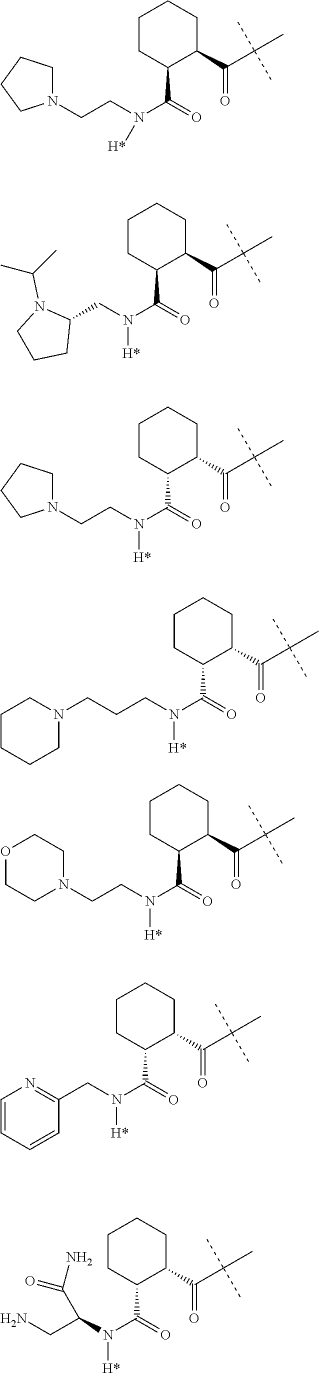

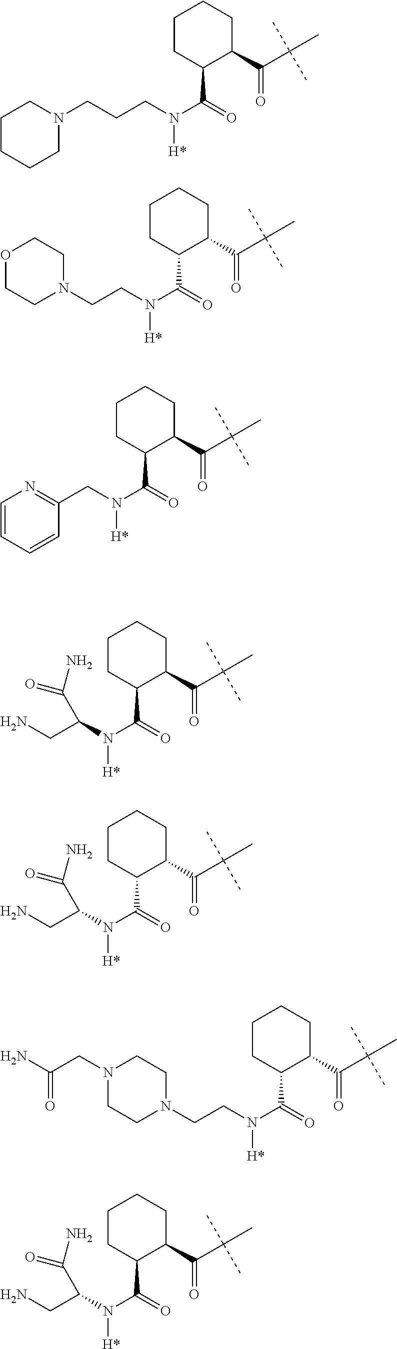

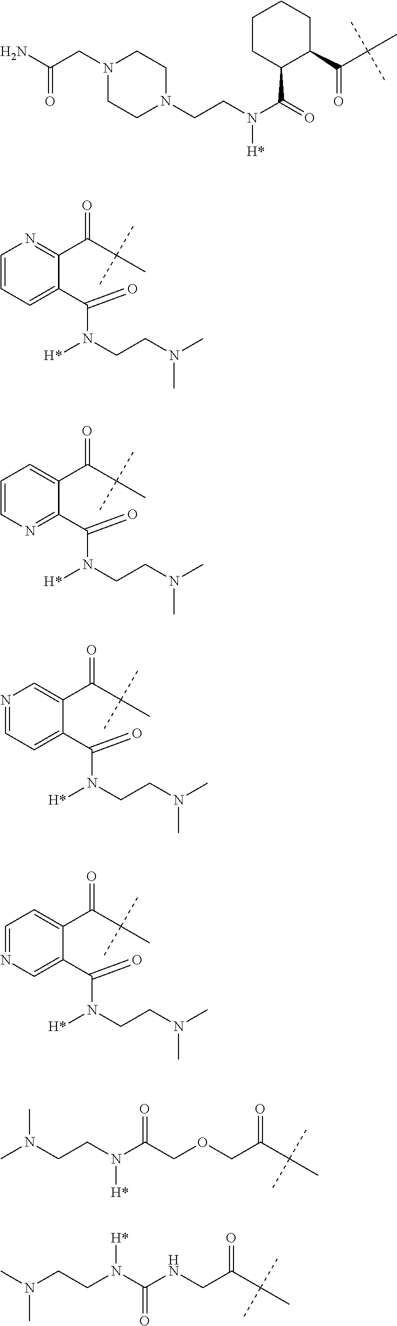

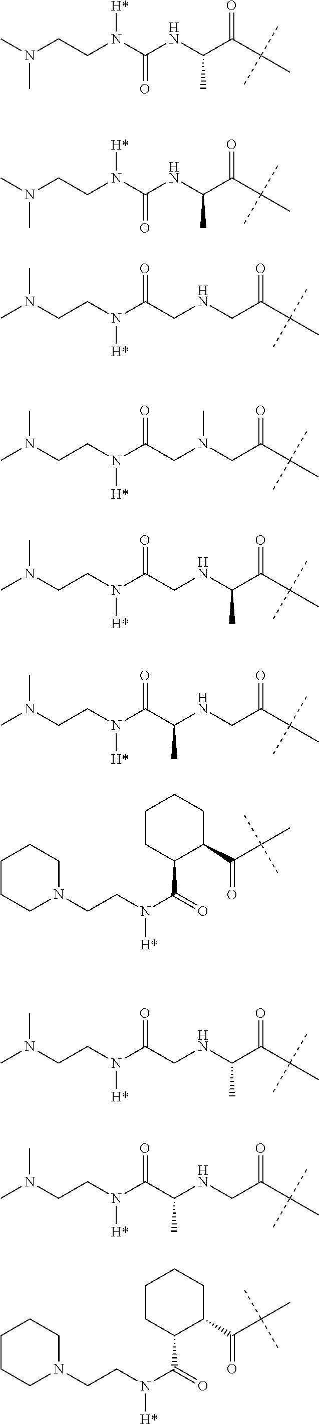

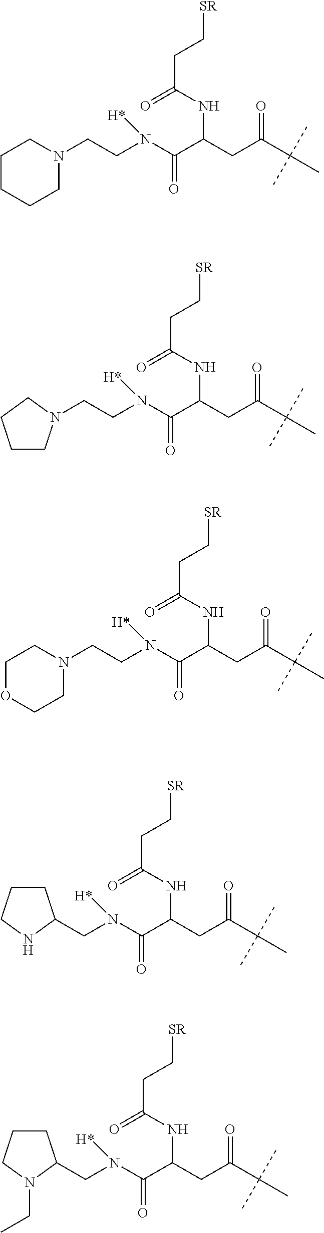

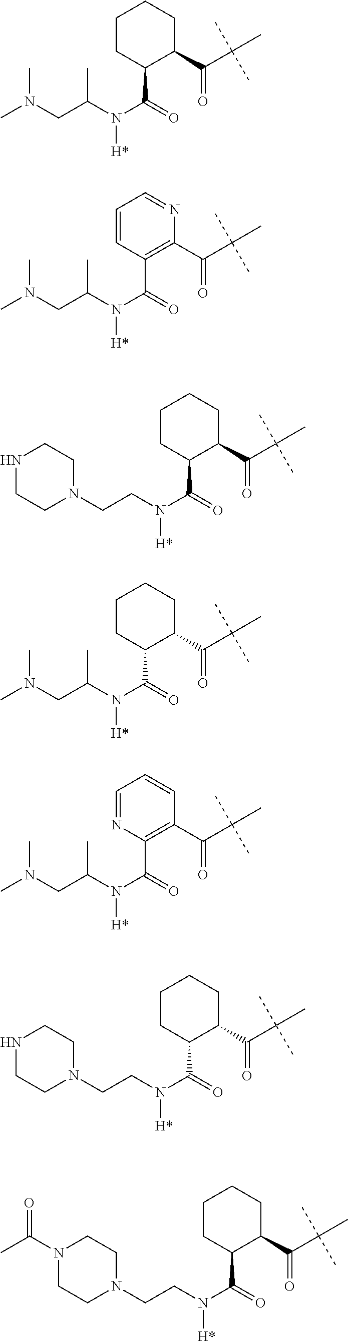

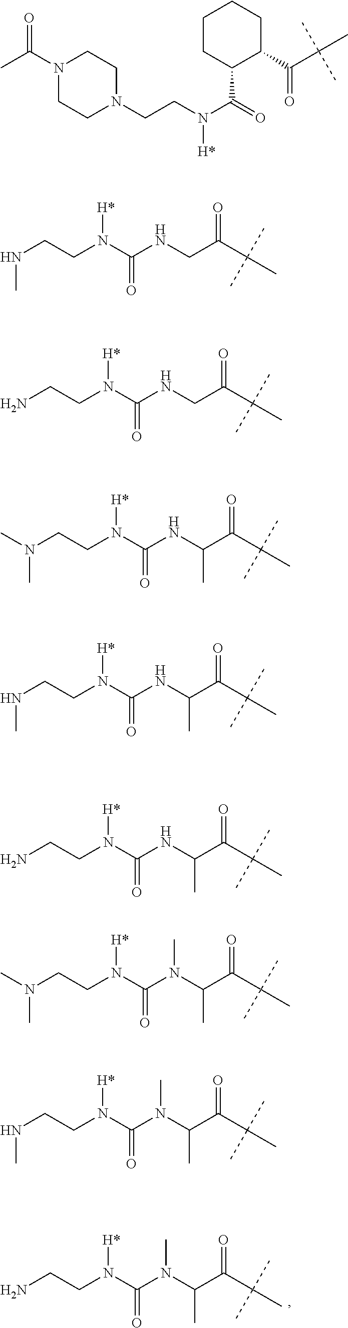

wherein R has the meaning as indicated above.

At least one (up to four) hydrogen is replaced by a group L²-Z. In case more than one group L²-Z is present each L² and each Z can be selected independently. Preferably, only one group L²-Z is present.

In general, S⁰ can be substituted with L²-Z at any position apart from the replacement of the hydrogen marked with an asterisk in the formulae above. Preferably, one to four of the hydrogen given by R, R¹ to R⁸ directly or as hydrogen of the C₁₋₄ alkyl or further groups and rings given by the definition of R and R¹ to R⁸ are replaced by L²-Z.

Furthermore, S⁰ may be optionally further substituted. In general, any substituent may be used as far as the cleavage principle is not affected.

Preferably, one or more further optional substituents are independently selected from the group consisting of halogen; CN; COOR⁹; OR⁹; C(O)R⁹; C(O)N(R⁹R^{9a}); S(O)₂N(R⁹R^{9a}); S(O)N(R⁹R^{9a}); S(O)₂R⁹; S(O)R^{9; N(R}⁹)S(O)₂N(R^{9a}R^{9b}); SR⁹; N(R⁹R^{9a}); NO₂; OC(O)R⁹; N(R⁹)C(O)R^{9a}); N(R⁹)S(O)₂R^{9a}; N(R⁹)S(O)R^{9a}; N(R⁹)C(O)OR^{9a}; N(R⁹)C(O)N(R^{9a}R^{9b}); OC(O)N(R⁹R^{9a}); T; C₁₋₅₀ alkyl; C₂₋₅₀ alkenyl; or C₂₋₅₀ alkynyl, wherein T; C₁₋₅₀ alkyl; C₂₋₅₀ alkenyl; and C₂₋₅₀ alkynyl are optionally substituted with one or more R¹⁰, which are the same or different and wherein C₁₋₅₀ alkyl; C₂₋₅₀ alkenyl; and C₂₋₅₀ alkynyl are optionally interrupted by one or more groups selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R¹¹R^{11a});

R⁹, R^{9a}, R^{9b} are independently selected from the group consisting of H; T; and C₁₋₅₀ alkyl; C₂₋₅₀ alkenyl; or C₂₋₅₀ alkynyl, wherein T; C₁₋₅₀ alkyl; C₂₋₅₀ alkenyl; and C₂₋₅₀ alkynyl are optionally substituted with one or more R¹⁰, which are the same or different and wherein C₁₋₅₀ alkyl; C₂₋₅₀ alkenyl; and C₂₋₅₀ alkynyl are optionally interrupted by one or more groups selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R₁₁R₁₁ₐ);

T is selected from the group consisting of phenyl; naphthyl; indenyl; indanyl; tetralinyl; C₃₋₁₀ cycloalkyl; 4 to 7 membered heterocyclyl; or 9 to 11 membered heterobicyclyl, wherein T is optionally substituted with one or more R¹⁰, which are the same or different;

R¹⁰ is halogen; CN; oxo (═O); COOR¹²; OR¹²; C(O)R¹²; C(O)N(R¹²R^{12a}); S(O)₂N(R¹²R^{12a}); S(O)N(R¹²R^{12a}); S(O)₂R¹²; S(O)R¹²; N(R¹²)S(O)₂N(R^{12a}R^{12b}); SR¹², N(R¹²R^{12a}); NO₂; OC(O)R¹²; N(R¹²)C(O)R^{12a}; N(R¹²)S(O)₂R^{12a}; N(R¹²)S(O)R^{12a}; N(R¹²)C(O)OR^{12a}; N(R¹²)C(O)N(R^{12a}R^{12b}); OC(O)N(R¹²R^{12a}); or C₁₋₆ alkyl, wherein C₁₋₆ alkyl is optionally substituted with one or more halogen, which are the same or different;

R¹¹, R^{11a}, R¹², R^{12a}, R^{12b} are independently selected from the group consisting of H; or C₁₋₆ alkyl, wherein C₁₋₆ alkyl is optionally substituted with one or more halogen, which are the same or different.

The term “interrupted” means that between two carbons a group is inserted or at the end of the carbon chain between the carbon and hydrogen.

L² is a single chemical bond or a spacer. In case L² is a spacer, it is preferably defined as the one or more optional substituents defined above, provided that L² is substituted with Z.

Accordingly, when L² is other than a single chemical bond, L²-Z is COOR⁹; OR⁹; C(O)R⁹; C(O)N(R⁹R^{9a}); S(O)₂N(R⁹R^{9a}); S(O)N(R⁹R^{9a}); S(O)₂R⁹; S(O)R⁹; N(R⁹)S(O)₂N(R^{9a}R^{9b}); SR⁹; N(R⁹R^{9a}); OC(O)R⁹; N(R⁹)C(O)R^{9a}; N(R⁹)S(O)₂R^{9a}; N(R⁹)S(O)R^{9a}; N(R⁹)C(O)OR^{9a}; N(R⁹)C(O)N(R^{9a}R^{9b}); OC(O)N(R⁹R^{9a}); T; C₁₋₅₀ alkyl; C₂₋₅₀ alkenyl; or C₂₋₅₀ alkynyl, wherein T; C₁₋₅₀ alkyl; C₂₋₅₀ alkenyl; and C₂₋₅₀ alkynyl are optionally substituted with one or more R¹⁰, which are the same or different and wherein C₁₋₅₀ alkyl; C₂₋₅₀ alkenyl; and C₂₋₅₀ alkynyl are optionally interrupted by one or more groups selected from the group consisting of -T-, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R¹¹R^{11a});

R⁹, R^{9a}, R^{9b} are independently selected from the group consisting of H; Z; T; and C₁₋₅₀ alkyl; C₂₋₅₀ alkenyl; or C₂₋₅₀ alkynyl, wherein T; C₁₋₅₀ alkyl; C₂₋₅₀ alkenyl; and C₂₋₅₀ alkynyl are optionally substituted with one or more R¹⁰, which are the same or different and wherein C₁₋₅₀ alkyl; C₂₋₅₀ alkenyl; and C₂₋₅₀ alkynyl are optionally interrupted by one or more groups selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R¹¹R^{11a});

T is selected from the group consisting of phenyl; naphthyl; indenyl; indanyl; tetralinyl; C₃₋₁₀ cycloalkyl; 4 to 7 membered heterocyclyl; or 9 to 11 membered heterobicyclyl, wherein t is optionally substituted with one or more R¹⁰, which are the same or different;

R¹⁰ is Z; halogen; CN; oxo (═O); COOR¹²; OR¹²; C(O)R¹²; C(O)N(R¹²R^{12a}); S(O)₂N(R¹²R^{12a}); S(O)N(R¹²R^{12a}); S(O)₂R¹²; S(O)R¹²; N(R¹²)S(O)₂N(R^{12a}R^{12b}); SR¹²; N(R¹²R^{12a}); NO₂; OC(O)R¹²; N(R¹²)C(O)R^{12a}; N(R¹²)S(O)₂R^{12a}; N(R¹²)S(O)R^{12a}; N(R¹²)C(O)OR^{12a}; N(R¹²)C(O)N(R^{12a}R^{12b}); OC(O)N(R¹²R^{12a}); or C₁₋₆ alkyl, wherein C₁₋₆ alkyl is optionally substituted with one or more halogen, which are the same or different;

R¹¹, R^{11a}, R¹², R^{12a}, R^{12b} are independently selected from the group consisting of H; Z; or C₁₋₆ alkyl, wherein C₁₋₆ alkyl is optionally substituted with one or more halogen, which are the same or different;

provided that one of R⁹, R^{9a}, R^{9b}, R¹⁰, R¹¹, R^{11a}, R¹², R^{12a}, R^{12b} is Z.

Preferably, the pharmaceutical composition of the present invention comprises a prodrug, which has a residual activity in an in vitro antiviral assay of less than 5%. More preferably, the in vitro antiviral residual activity of the conjugate is less than 3%, and even more preferred the in vitro antiviral residual activity of the conjugate is less than 1%. The in vitro antiviral residual activity can be measured as described in Example 6.

Another aspect of the present invention is a water-soluble polymeric carrier linked prodrug as defined herein.

The pharmaceutical composition and the prodrug according to the present invention are useful in the technical fields, where also interferon alpha is used.

Exemplary conditions which can be treated with interferon include but are not limited to cell proliferation disorders, in particular cancer (e.g., hairy cell leukemia, Kaposi's sarcoma, chronic myelogenous leukemia, multiple myeloma, basal cell carcinoma and malignant melanoma, ovarian cancer, cutaneous T cell lymphoma), and viral infections. Without limitation, treatment with interferon may be used to treat conditions which would benefit from inhibiting the replication of interferon-sensitive viruses. Viral infections which may be treated in accordance with the invention include hepatitis A, hepatitis B, hepatitis C, other non-A/non-B hepatitis, herpes virus, Epstein-Barr virus (EBV), cytomegalovirus (CMV), herpes simplex, human herpes virus type 6 (HHVL6), papilloma, poxvirus, picornavirus, adenovirus, rhinovirus, human T lymphotropic virus-type 1 and 2 (HTLV-1/-2), human rotavirus, rabies, retroviruses including human immunodeficiency virus (HIV), encephalitis and respiratory viral infections.

Accordingly, another aspect of the present invention is a pharmaceutical composition of the present invention or a prodrug of the present invention for use in a method of treating, controlling, delaying or preventing a condition that can benefit from interferon alpha treatment. Preferred conditions are mentioned above.

Accordingly, another aspect of the present invention is a method for treating, controlling, delaying or preventing in a mammalian patient in need of the treatment of a condition that can benefit from interferon alpha treatment, wherein the method comprises the administration to said patient a therapeutically effective amount of the pharmaceutical composition of any of the present invention or a prodrug of the present invention. Preferred conditions are mentioned above.

Preferably, the treatment of a virally infected patient results in a reduced viral relapse rate compared to a drug conjugate of a permanently PEGylated interferon alpha. Relapse rate is defined as percentage of patients with undetectable HCV-RNA at the end of the treatment period and detectable HCV-RNA at 6 months post-treatment, as measured by standard analytical tests.

Preferably, the administration results in an increased volume of distribution over permanently PEGylated interferon alpha. Volume of distribution is defined as the theoretical volume of fluid into which the total drug administered would have to be diluted to produce the concentration measured in the plasma.

The composition of polymeric carrier-linked prodrug of interferon alpha may be provided as a liquid composition or as a dry composition.

In case of dry compositions, suitable methods of drying are, for example, spray-drying and lyophilization (freeze-drying). Preferably, the pharmaceutical composition of polymeric carrier-linked interferon alpha prodrug is dried by lyophilization.

Preferably, the polymeric carrier-linked interferon alpha prodrug in either liquid or dry composition is sufficiently dosed in the composition to provide therapeutically effective amount of interferon for one week or longer in one application. More preferably, one application of the polymeric carrier-linked interferon alpha prodrug is sufficient for one to four weeks.

The pharmaceutical composition of polymeric carrier-linked interferon alpha prodrug according to the present invention, whether in dry or liquid form or in another form, contains one or more excipients.

Excipients used in parenteral compositions may be categorized as buffering agents, isotonicity modifiers, preservatives, stabilizers, anti-adsorption agents, oxidation protection agents, viscosifiers/viscosity enhancing agents, or other auxiliary agents. In some cases, these ingredients may have dual or triple functions. The one or more than one excipient is selected from the groups consisting of:
(i) Buffering agents: physiologically tolerated buffers to maintain pH in a desired range, such as sodium phosphate, bicarbonate, succinate, histidine, citrate and acetate, sulphate, nitrate, chloride, pyruvate. Antacids such as Mg(OH)₂ or ZnCO₃ may be also used. Buffering capacity may be adjusted to match the conditions most sensitive to pH stability
(ii) Isotonicity modifiers: to minimize pain that can result from cell damage due to osmotic pressure differences at the injection depot. Glycerin and sodium chloride are examples. Effective concentrations can be determined by osmometry using an assumed osmolality of 285-315 mOsmol/kg for serum
(iii) Preservatives and/or antimicrobials: multidose parenteral preparations require the addition of preservatives at a sufficient concentration to minimize risk of patients becoming infected upon injection and corresponding regulatory requirements have been established. Typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride
(iv) Stabilizers: Stabilisation is achieved by strengthening of the protein-stabilising forces, by destabilisation of the denatured stater, or by direct binding of excipients to the protein. Stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives. In addition, oligomers or polymers such as cyclodextrins, dextran, dendrimers, PEG or PVP or protamine or HSA may be used
(v) Anti-adsorption agents: Mainly ionic or iron-ionic surfactants or other proteins or soluble polymers are used to coat or adsorb competitively to the inner surface of the composition's or composition's container. E.g., poloxamer (Pluronic F-68), PEG dodecyl ether (Brij 35), polysorbate 20 and 80, dextran, polyethylene glycol, PEG-polyhistidine, BSA and HSA and gelatines. Chosen concentration and type of excipient depends on the effect to be avoided but typically a monolayer of surfactant is formed at the interface just above the CMC value
(vi) Lyo- and/or cryoprotectants: During freeze- or spray drying, excipients may counteract the destabilising effects caused by hydrogen bond breaking and water removal. For this purpose sugars and polyols may be used but corresponding positive effects have also been observed for surfactants, amino acids, non-aqueous solvents, and other peptides. Trehalose is particulary efficient at reducing moisture-induced aggregation and also improves thermal stability potentially caused by exposure of protein hydrophobic groups to water. Mannitol and sucrose may also be used, either as sole lyo/cryoprotectant or in combination with each other where higher ratios of mannitol: sucrose are known to enhance physical stability of a lyophilized cake. Mannitol may also be combined with trehalose. Trehalose may also be combined with sorbitol or sorbitol used as the sole protectant. Starch or starch derivatives may also be used
(vii) Oxidation protection agents: antioxidants such as ascorbic acid, ectoine, methionine, glutathione, monothioglycerol, morn, polyethylenimine (PEI), propyl gallate, vitamin E, chelating agents such aus citric acid, EDTA, hexaphosphate, thioglycolic acid
(viii) Viscosifiers or viscosity enhancers: retard settling of the particles in the vial and syringe and are used in order to facilitate mixing and resuspension of the particles and to make the suspension easier to inject (i.e., low force on the syringe plunger). Suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone. Such block copolymers as well as the abovementioned poloxamers may exhibit reverse thermal gelation behavior (fluid state at room temperature to facilitate administration and gel state above sol-gel transition temperature at body temperature after injection).
(ix) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue. A spreading agent such as but not limited to hyaluronidase temporarily decreases the viscosity of the extracellular matrix and promotes diffusion of injected drugs.
(x) Other auxiliary agents: such as wetting agents, viscosity modifiers, antibiotics, hyaluronidase.

Acids and bases such as hydrochloric acid and sodium hydroxide are auxiliary agents necessary for pH adjustment during manufacture

It is preferred, that a dry composition comprises one or more preservative and/or antimicrobial.

In one embodiment of the present invention, the dry or liquid or other form of composition of polymeric carrier-linked interferon alpha prodrug is provided as a single dose, meaning that the container in which it is supplied contains one pharmaceutical dose.

In another aspect of the present invention the liquid or dry or other form of composition is provided as a multiple dose composition, meaning that the container in which it is supplied contains more than one pharmaceutical dose. Such multiple dose composition of polymeric carrier-linked interferon alpha prodrug can either be used for different patients in need thereof or is intended for use in one patient, wherein the remaining doses are stored after the application of the first dose until needed.

In another aspect of the present invention the liquid or dry or other form of composition is comprised in a container.

Suitable containers for liquid compositions are, for example, syringes, vials, vials with stopper and seal, ampouls, and cartridges. In particular, the liquid compositions according to the present invention are provided in a syringe.

Suitable containers for dry compositions are, for example, syringes, dual-chamber syringes, vials, vials with stopper and seal, ampouls, and cartridges. In particular, the dry composition according to the present invention is provided in a first chamber of the dual-chamber syringe and reconstitution solution is provided in a second chamber of the dual-chamber syringe.

Prior to applying the dry composition polymeric carrier-linked interferon alpha prodrug to a patient in need thereof, the dry composition is reconstituted. Reconstitution can take place in the container in which the dry composition of polymeric carrier-linked interferon alpha prodrug is provided, such as in a vial, syringe, dual-chamber syringe, ampoule, and cartridge. Reconstitution is done by adding a predefined amount of reconstitution solution to the dry composition. Reconstitution solutions are sterile liquids, such as water or buffer, which may contain further additives, such as preservatives and/or antimicrobials, such as, for example, benzylalcohol and cresol. Preferably, the reconstitution solution is sterile water.

An additional aspect of the present invention relates to the method of administration of a reconstituted or liquid polymeric carrier-linked interferon alpha prodrug composition. The polymeric carrier-linked interferon alpha prodrug composition can be administered by methods of injection or infusion, including intradermal, subcutaneous, intramuscular, intravenous, intraosseous, and intraperitoneal. Preferably, the polymeric carrier-linked interferon alpha prodrug prodrug is administered subcutaneously.

A further aspect is a method of preparing a reconstituted composition comprising a therapeutically effective amount of a polymeric carrier-linked interferon alpha prodrug, and optionally one or more pharmaceutically acceptable excipients, wherein the interferon alpha is transiently linked to a polymeric carrier, the method comprising the step of
contacting the dry composition of the present invention with a reconstitution solution.

Another aspect is a reconstituted composition comprising a therapeutically effective amount of a polymeric carrier-linked interferon alpha prodrug, and optionally one or more pharmaceutically acceptable excipients, wherein the interferon alpha is transiently linked to a polymer carrier as described above.

Another aspect of the present invention is the method of manufacturing a liquid composition of polymeric carrier-linked interferon alpha prodrug. In one embodiment, such liquid composition is made by
(i) admixing the polymeric carrier-linked interferon alpha prodrug with one or more excipients,
(ii) transfering amounts equivalent to single or multiple doses into a suitable container, and
(iii) sealing the container.

Suitable containers are syringes, vials, vials with stopper and seal, ampouls, and cartridges.

Another aspect of the present invention is the method of manufacturing a dry composition of polymeric carrier-linked interferon alpha prodrug. In one embodiment, such dry composition is made by
(i) admixing the polymeric carrier-linked interferon alpha prodrug with one or more excipients,
(ii) transfering amounts equivalent to single or multiple doses into a suitable container,
(iii) drying the composition in said container, and
(iv) sealing the container.

Suitable containers are syringes, dual-chamber syringes, vials, vials with stopper and seal, ampouls, and cartridges.

Another aspect is a kit of parts for a dry composition according to the present invention. When the administration device is simply a hypodermic syringe then the kit may comprise the syringe, a needle and a container comprising the dry polymeric carrier-linked interferon alpha prodrug composition for use with the syringe and a second container comprising the reconstitution solution. In more preferred embodiments, the injection device is other than a simple hypodermic syringe and so the separate container with reconstituted polymeric carrier-linked interferon alpha prodrug is adapted to engage with the injection device such that in use the liquid composition in the container is in fluid connection with the outlet of the injection device. Examples of administration devices include but are not limited to hypodermic syringes and pen injector devices. Particularly preferred injection devices are the pen injectors in which case the container is a cartridge, preferably a disposable cartridge.

A preferred kit of parts for a dry composition comprises a needle and a container containing the composition according to the present invention and optionally further containing a reconstitution solution, the container being adapted for use with the needle. Preferably, the container is a dual-chamber syringe.

Another aspect is a kit of parts for a liquid composition according to the present invention. When the administration device is simply a hypodermic syringe then the kit may comprise a container with the liquid composition and a needle for use with the container.

In another aspect, the invention provides a cartridge containing composition of polymeric carrier-linked interferon alpha prodrug, whether in liquid or dry or other form, as hereinbefore described for use with a pen injector device. The cartridge may contain a single dose or multiplicity of doses of polymeric carrier-linked interferon alpha prodrug.

Another aspect of the present invention is a prodrug of the present invention or a pharmaceutical composition of the present invention for use as a medicament.

Another aspect of the present invention is a prodrug of the present invention or a pharmaceutical composition of the present invention for use in a method of treating or preventing diseases or disorders which can be treated by interferon alpha as described above.

Another aspect of the present application is the combination of a polymeric carrier-linked interferon alpha prodrug of the present invention with one or more other biologically active moieties. Such other biologically active moieties may either be used in their free form or in the form of prodrugs.

If the carrier-linked interferon alpha prodrug is used for the treatment of hepatitis C virus (HCV), any compound with anti-HCV activity may be suitable for such a combination prodrug, combination composition or combination treatment. Such compound is effective to inhibit the function of a target which may be selected from the group consisting of HCV metalloprotease, HCV serine protease, HCV polymerase, HCV helicase, HCV NS4B protein, HCV entry, HCV assembly, HCV egress, HCV NS5A protein, IMPDH and a nucleoside analog.

More specifically, suitable biologically active moieties may be selected from the following groups:
(i) Nucleoside antimetabolites: such as broad spectrum anti viral compounds, including Ribavirin and Viramidine.
(ii) Small molecule antivirals: such as HCV protease and polymerase inhibitors such as NS5B polymerase inhibitors and NS3 protease. Examples of compounds in clinical development are for example Telaprevir, Boceprevir, GS 9190, TMC-435350, R7227/ITMN-191, BI201335, BMS-790052 and R-7128.
(iii) Immunomodulators: such as SCV-07, Civacir, Alinia, Zadaxin, Bavituximab, IPH1101 and CYT107
(iv) Therapeutic vaccines: such as IC-41, GI-5005 and ChronVac-C
(v) Host enzyme inhibitors: such as Celgosivir, Debio-025 and NIM811

The carrier-linked interferon alpha prodrug can be used for the treatment of oncological indications. In one embodiment, the composition may optionally contain one or more additional anti-cancer compounds such as, but not limited to, allopurinol sodium, cladribine, cytarabine, darcarbazine, doxorubicin, daunorubicin, etoposide, floxuridine, fluorouracil, ifosfamide, leucovorin calcium, leuprolide acetate, mesna, methotrexate, mitomycin, mitoxantrone hydroclhloride, octreotide acetate, pamidronatye disodium, thiotepa, vinorelbine, bleomycin, dacarbazine, vincristine, vinblastine, paclitaxel, docetaxel, cisplatin, carboplatin, actinomycin D, and/or combined with any of the following: surgery, or radiation, or hormonal treatments, or specific inhibitors, or antibodies, or antibody fragments, or vaccines, or small molecule drugs, or other cytokines, or biological molecules orantisense, or gene therapy.

Oncological indications to be treated with a carrier-linked interferon alpha prodrug may include: acute myeloid leukemia, adrenocortical carcinoma, anal cancer, appendix cancer, astrocytoma, bile duct cancer, bladder cancer, bone cancer, osteosarcoma/malignant fibrous histiocytoma, brain stem glioma, brain tumours, breast cancer, bronchial adenomas/carcinoids, Burkitt lymphoma, carcinoid tumour, central nervous system lymphoma, cerebellar astrocytoma, cervical cancer, chronic lymphocytic leukemia, chronic myelogenous leukemia, chronic myeloproliferative disorders, colon cancer, cutaneous T-cell lymphoma, endometrial cancer, ependymoma, esophageal cancer, extracranial germ cell tumour, extragonadal germ cell tumours, extrahepatic bile duct cancer, eye cancer, intraocular melanoma, eye cancer, retinoblastoma, gallbladder cancer, gastric (stomach) cancer, gastrointestinal carcinoid tumour, gastrointestinal stromal tumour (GIST), germ cell tumour, extracranial, germ cell tumour, extragonadal, germ cell tumour, ovarian, gestational trophoblastic tumour, glioma, adult glioma, childhood brain stem glioma, childhood cerebral astrocytoma, childhood visual pathway and hypothalamic, hairy cell leukemia, head and neck cancer, hepatocellular (liver) cancer, adult (primary), Hodgkin lymphoma, hypopharyngeal Cancer, hypothalamic and visual pathway glioma, childhood, intraocular melanoma, islet cell carcinoma (endocrine pancreas), Kaposi sarcoma, kidney (renal cell) cancer, laryngeal cancer, leukemia, acute lymphoblastic, leukemia, acute myeloid, leukemia, chronic lymphocytic, leukemia, chronic myelogenous, leukemia, hairy cell, lip and oral cavity cancer, liver cancer, lung cancer, non-small cell, lung cancer, small cell, lymphoma, AIDS-related, macroglobulinemia, Waldenstrom, malignant fibrous histiocytoma of bone/osteosarcoma, medulloblastoma, melanoma, Merkel cell carcinoma, mesothelioma, metastatic squamous neck cancer with occult primary, mouth cancer, multiple endocrine neoplasia syndrome, multiple myeloma/plasma cell neoplasm, myelogenous leukemia, chronic, myeloid leukemia, acute, myeloma, nasal cavity and paranasal sinus cancer, nasopharyngeal cancer, neuroblastoma, non-Hodgkin lymphoma, oral cancer, oral cavity cancer, lip and oropharyngeal cancer, ovarian cancer, pancreatic cancer, parathyroid cancer, penile cancer, pharyngeal cancer, pheochromocytoma, pineoblastoma and supratentorial primitive neuroectodermal tumours, pituitary tumour, plasma cell neoplasm/multiple myeloma, pleuropulmonary blastoma, prostate cancer, rectal cancer, renal cell (kidney) cancer, renal pelvis and ureter transitional cell cancer, retinoblastoma, rhabdomyosarcoma, salivary gland cancer, sarcoma, Kaposi's sarcoma, soft tissue, sarcoma, uterine, skin cancer (nonmelanoma), small intestine cancer, testicular cancer, throat cancer, thymoma and thymic carcinoma, thyroid cancer, urethral cancer, vaginal cancer, vulvar cancer, Waldenstrom macroglobulinemia, Wilm's tumour and any other oncological indication which may be treated by a type I interferon.

However, it is understood that the use of a carrier-linked interferon alpha prodrug according to the present invention is not limited to HCV and oncology and that the present invention also covers the treatment or prevention of any disease or disorder which can be treated by interferon alpha.

It is also understood that any combination of one or more other biologically active moieties is covered in this invention.

### EXAMPLES

Methods

Automated Flash Chromatography

Automated Flash Chromatography was performed on a Biotage “Isolera one” purification system. Products were detected and collected at 254 and 280 nm.

Analytical and Preparative RP-HPLC

Analytical RP-HPLC/ESI-MS was performed on Waters equipment consisting of a 2695 sample manager, a 2487 Dual Absorbance Detector, and a ZQ 4000 ESI instrument equipped with a 5 μm Reprosil Pur 300 Å ODS-3 columns (75×1.5 mm) (Dr. Maisch, Ammerbuch, Germany; flow rate: 350 μl/min, typical gradient: 10-90% MeCN in water, 0.05% TFA over 5 min) and spectra were, if necessary, interpreted by Waters software MaxEnt.

Analytical HPLC was performed on a Agilent 1200, Agilent Technologies (comprising G1379B degasser, G1312A binary pump, G1329A thermostatted autosampler, G1316A column oven, G1365D multi wavelength detector equipped with a waters Acquity BEH300 C18 column (1.7 μm; 2.1×50 mm).

RP-UPLC/ESI-MS was performed on Waters/Thermo equipment consisting of a Waters Acquity UPLC with an Acquity PDA detector coupled to a Thermo LTQ Orbitrap Discovery high resolution/high accuracy mass spectrometer equipped with a C18 RP column (2.1×50 mm, 300 Å, 1.7 μm, Flow: 0.25 mL/min (max back pressure 270 bar); solvent A: UP-H20, 0.025% TFA, solvent B: 100% MeCN.

For preparative RP-HPLC a Waters 600 controller and a 2487 Dual Absorbance Detector was used equipped with the following columns (Reprosil Pur 300 Å ODS-3)

A): 100×20 mm, 10 mL/min flow rate, typical gradient: 10-90% MeCN in water, 0.1% TFA over 11 min

or

B): 100×40 mm (10 μm particles), 40 mL/min flow rate, typical gradient: 10-90% MeCN in water, 0.1% TFA over 11 min

Cation Exchange Chromatography

The purification of conjugates by cation exchange chromatography was performed using a ÄKTA explorer system (GE Healthcare) or an Amersham Bioscience ÄKTA basic system equipped with a Macrocap SP column (6 ml). The respective conjugate in 20 mM acetate buffer, pH 4 (buffer A) was applied to the column that was pre-equilibrated in (buffer A). The column was washed with three column volumes of buffer A to remove any unreacted PEG reagent. Conjugates were eluated using a gradient of 0-25% buffer B (20 mM sodium acetate, 1 M NaCl pH 4.5) over 20 CV followed by 25-80% buffer B over 3 CV. The eluent was monitored by detection at 280 and 215 nm.

Analytical Size Exclusion Chromatography

Size exclusion chromatography (SEC) was performed using an Amersham Bioscience AEKTAbasic system or an ÄKTA explorer system (GE Healthcare) equipped with a Superdex200 10/300 column (Amersham Bioscience/GE Healthcare) or a Sepharose 6 column and 15 mM sodium phosphate, 135 mM NaCl, pH 7.4 as mobile phase. The flow rate for both columns was 0.75 ml/min and the eluated interferon and polymer-interferon conjugates were detected at 215 and 280 nm.

Buffer Exchange

Buffer exchange was performed using an Amersham Bioscience AEKTAbasic system or an ÄKTA explorer system (GE Healthcare) equipped with a HiPrep 26/10 Desalting column or a HiTrap Desalting column.

Concentration of the PEG-Linker-IFN Conjugates

Concentration was performed using an AMIOCON Stirred Ultrafiltration Cell (model 8003 or model 8010) equipped with a regenerated cellulose membrane (MWCO: 10.000-100.000).

Activity Determination of pfp-Activated mPEG-Linker Reagents

A defined amount of pfp-activated mPEG-linker reagent (3-5 mg) was dissolved in 100 μl H₂O. 10 μl 0.5 M NaOH were added and the reaction mixture was reacted for 60 min at 40° C. 1.5 μl TFA was added and 10% of this mixture was analyzed by analytical RP-HPLC. The chromatograms were recorded at 260 and 280 nm. The peak corresponding to pentafluorophenol was integrated. Determined values were compared with an appropriate calibration curve generated by analyzing defined amounts of pfp by analytical RP-HPLC and integration of chromatograms recorded at 260 and 280 nm.

SDS-PAGE Analysis

PEG-interferon conjugates were analyzed using NuPAGE® Novex Tris-Acetate gels (1.0 mm thick, 12 lanes) with NuPAGE Tris-Acetate SDS-Running Buffer or NuPAGE® Novex Bis-Tris gels (1.0 mm thick, 12 lanes) with NuPAGE MOPS SDS-Running Buffer, HiMark™ Pre-Stained High Molecular Weight Protein Standard and Simply Blue™ SafeStain (Invitrogen). In each lane 0.2-0.6 μg were applied and the electrophoresis and subsequent staining performed according to the supplier's protocol.

### Example 1

### Synthesis of Permanent Linker Reagent 11a and Transient Linker Reagent 11b

Synthesis of Compound 5

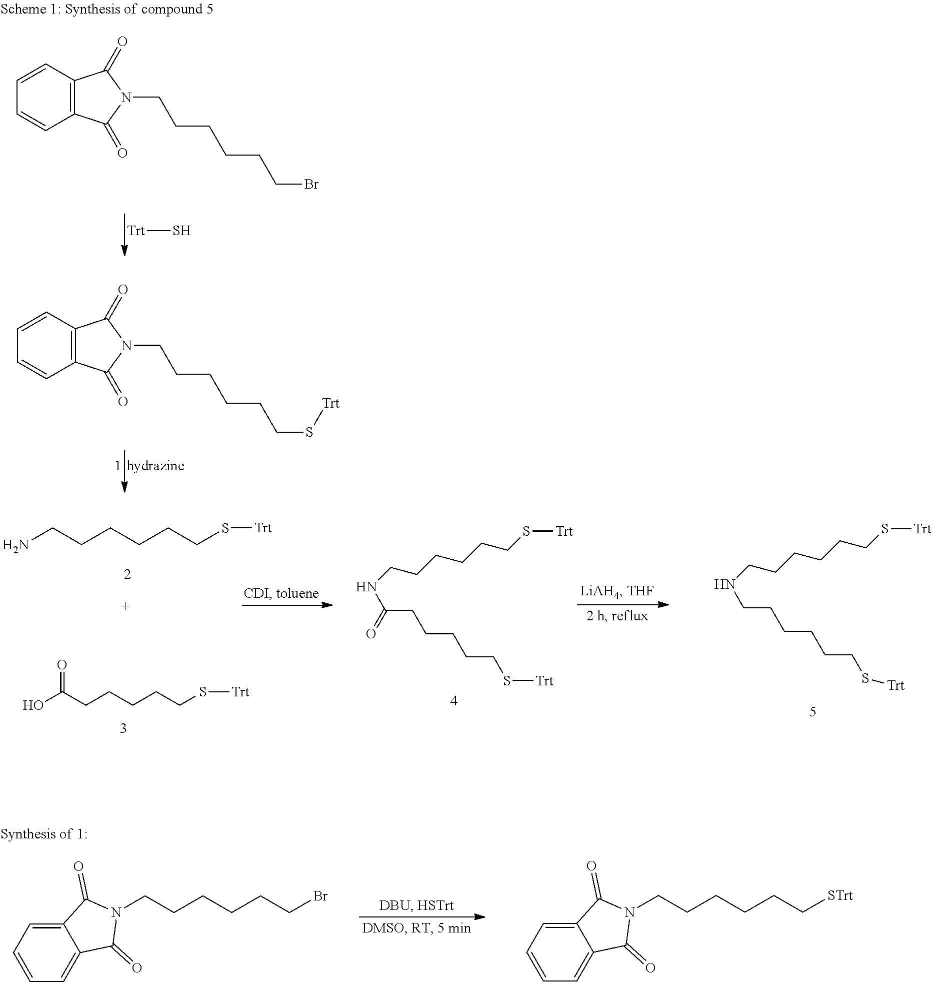

Under an atmosphere of nitrogen, triphenylmethanethiol (11.90 g, 43.08 mmol) was suspended in DMSO (40 ml). DBU (7.41 ml, 49.55 mmol) was added slowly, and the mixture was stirred at RT for 5 min. Solid 6-bromohexylphthalimide (13.32 g, 42.94 mmol) was added in several portions, and the mixture was allowed to react for approximately 15 min. The brown viscous solution was partitioned between EtOAc (700 ml) and 0.1 M HCl (200 ml). The aqueous phase was extracted with EtOAc (3×50 ml), and the combined organic fractions were washed with NaHCO₃ sat. (80 ml) and brine (80 ml), dried over MgSO₄, filtered and concentrated. The crude yellow oil was recrystallized from n-heptane/EtOAc 8:1 (ca. 250-300 ml).

Yield 13.3 g (26.4 mmol, 62%) as white solid.

Synthesis of 2:

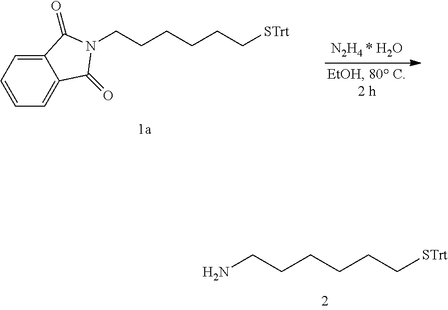

6-(S-Trityl)mercaptohexylphthalimid (14.27 g, 28.2 mmol) was suspended in EtOH (250 ml). Hydrazine hydrate (3.45 ml, 70.5 mmol) was added, and the mixture was heated to reflux for 2 h. The reaction mixture became clear, before a white precipitate formed. The mixture was filtered; the precipitate was washed with cold EtOH, and the filtrate was concentrated in vacuo. To the residual oil was added CHCl₃ (180 ml), and the resulting suspension was stirred at RT for 1.5 h. The mixture was filtered, the precipitate washed with cold CHCl₃, and the filtrate was extracted with H₂O (60 ml) and brine (60 ml), dried over MgSO₄, filtered and concentrated to give the crude amine, which was sufficiently pure for the next transformation.

2: Yield 10.1 g (26.87 mmol, 95% crude).

MS [M+H]⁺=367.21 g/mol (MW+H calculated=367.30 g/mol).

3 was purchased from NeoMPS (France)

Synthesis of 4:

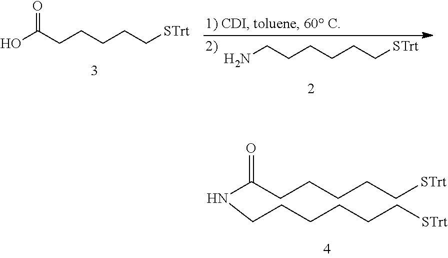

Under an atmosphere of nitrogen, tritylmercaptohexanoic acid 3 (8.46 g, 21.66 mmol) was dissolved in toluene (40 ml), and the solution was heated to 60° C. Carbonyldiimidazole (3.87 g, 23.87 mmol) was added in several portions, and the solution was stirred at 60° C. for 15 min. The amine 2 (8.15 g, 21.07 mmol) was added as a solution in toluene (20 ml), and the mixture was stirred at 60° C. for 2 h. After cooling to RT, the solution was partitioned between EtOAc (200 ml) and 0.1 M HCl (100 ml). The aqueous phase was extracted with EtOAc (3×30 ml), and the organic fractions were washed with NaHCO₃ sat. (75 ml) and brine (75 ml), dried over MgSO₄, filtered and concentrated. The crude product was adsorbed on celite and purified by flash chromatography (n-heptane/EtOAc 2:1 (v/v) to 1:1 (v/v)).

4: Yield 13.8 g (18.5 mmol, 85%) as slightly yellow foam.

R_{f}=0.5 (n-heptane/EtOAc 1:1).

MS [M+H]⁺=748.36 (MW+H calculated=748.28 g/mol).

Synthesis of 5:

Under nitrogen, amide 4 (4.82 g, 6.44 mmol) was dissolved in THF (25 ml), and a 1M solution of borane-THF complex (25 ml, 25 mmol) was added over the course of five minutes. The reaction mixture was stirred at RT for 21 h, before TLC analysis [n-heptane/EtOAc 1:1, Rf (amine-borane intermediate)=0.60] indicated complete consumption of starting material. After cooling to 0° C., excess borane was quenched with MeOH (ca. 4 ml). N,N′-dimethylethylenediamine (4.2 ml, 38.64 mmol) was added, and the mixture was brought to reflux for 2.5 h. After cooling to RT, the solvent was removed in vacuo, and the residue was dissolved in 100 ml of EtOAc. The solution was washed with 60 ml of H₂O. The aqueous phase was extracted with EtOAc (4×30 ml), and the combined organic fractions were washed with brine (60 ml), dried over Na₂SO₄, filtered and concentrated. The crude product was purified by flash chromatography (300 ml silica, CH₂Cl₂/MeOH 19:1 (v/v)+0.1% NEt₃). Product 5 was obtained as yellow oil.

5: Yield 3.71 g (5.048 mmol, 78%).

MS [M+H]⁺=734.38 (MW+H calculated=734.28 g/mol).

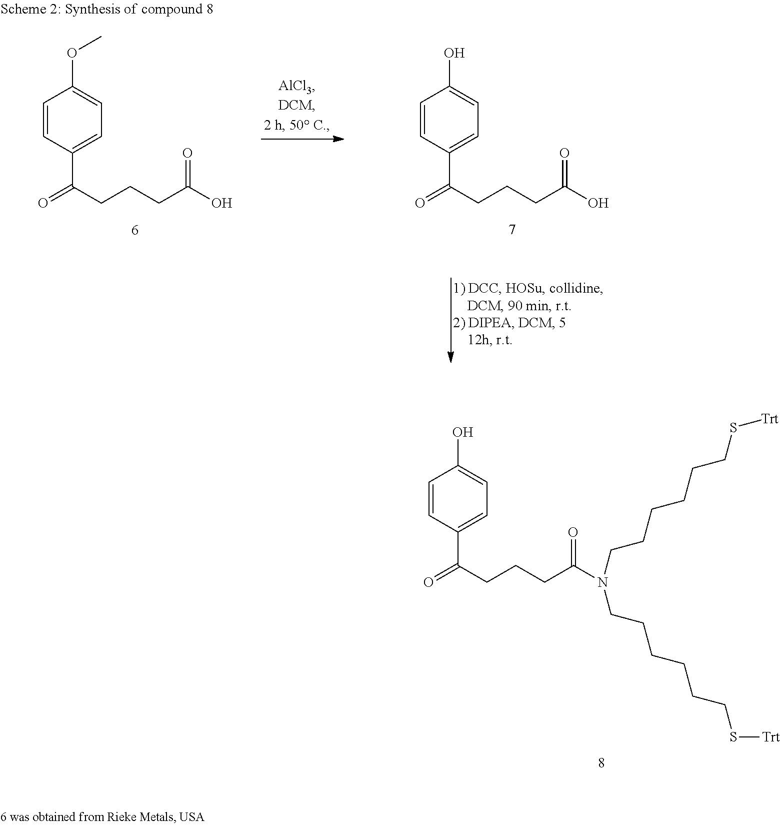

Synthesis of 7

AlCl₃ (9.0 mg, 68 mmol) was added to 6 (5.0 g, 23 mmol) in 1,2-dichloroethane (50 ml). The reaction mixture was stirred for 7 h at 85° C. During the reaction time, a highly viscous brown precipitate formed, which was broken into small pieces (3 times). The final dark-brown mixture was cooled to RT. Ice cold 1 N HCl (50 ml) was added, and the organic phase was diluted with EtOAc until the precipitate was completely dissolved (>400 ml). The phases were separated, and the aqueous phase was extracted with EtOAc (4×50 ml). The combined organic fractions were dried over Na₂SO₄, filtered and concentrated in vacuo to give a light red solid that was used in the next step without further purification.

7: Yield 4 g (19.1 mmol, 98%).

MS [M+H]⁺=209.1 g/mol (MW+H calculated=209.1 g/mol).

Synthesis of 8:

To a RT solution of 7 (4.66 g, 22.4 mmol) in CH₂Cl₂ (98 ml) were added dicyclohexylcarbodiimide (5.78 g, 28.0 mmol), HOSu (3.06 g, 26.6 mmol) and collidine (10.93 ml, 84 mmol). After 90 min, the reaction mixture was filtered (in order to remove the precipitated dicyclohexylurea) directly to amine 5, and DIPEA (9.75 ml, 56.0 mmol) was added. The mixture was stirred at RT for 1.5 h and subsequently diluted with EtOAc (400 ml). The solution was washed with 0.1 M HCl (200 mL), and the aqueous phase was extracted with EtOAc (3×50 ml). The combined organic fractions were washed with NaHCO₃ sat. (100 ml) and brine (100 ml), dried over MgSO₄, filtered and concentrated. The crude material was adsorbed on celite and purified by automated flash chromatography on silica in three portions (SNAP 100 g cartridge, flow 40 ml/min, solvent A: n-heptane, solvent B: EtOAc; gradient: 10% B (6 CV), 40% B (3.9 CV), 60% B (3.5 CV)).

8: Yield 7.58 g (8.20 mmol, 59%).

MS [M+H]⁺=924.46 g/mol (MW+H calculated=924.44 g/mol).

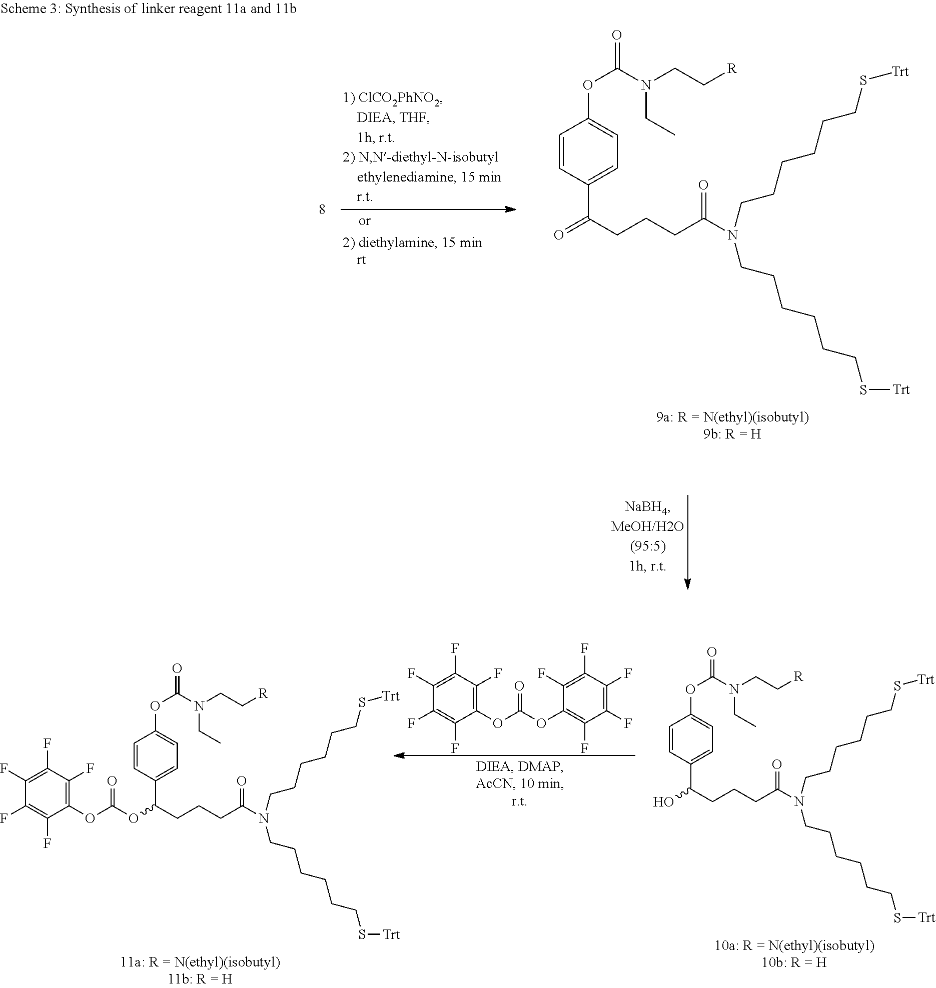

Synthesis of 9a

Synthesis of N,N′-diethyl, N-isobutyl-ethylenediamine by Solid Phase Synthesis

N,N′-diethyl-ethylenediamine (0.745 ml, 5.2 mmol) was dissolved in CH₂Cl₂ (7 ml) and added to the TCP-resin (1 g, 1.3 mmol/g, Novabiochem). The reaction mixture was gently shaken for 45 min before MeOH (1 ml) was added. After further 15 min the resin was washed 10 times with CH₂Cl₂ (2 ml) and dried under reduced pressure.

The TCP-resin bound to N,N′-diethyl-ethylenediamine (1 g) was washed 3 times with DMF (2 ml) and isobutyryl chloride (0.544 ml, 5.2 mmol) and pyridine (1.23 ml, 15.6 mmol) in DMF (5 ml) were added. The reaction mixture was shaken 2 h at RT. The resin was washed 10 times with DMF (2 ml) and CH₂Cl₂ (2 ml) and dried under reduced pressure.

The resin bound to N-ethyl-N-[2-(ethylamino)ethyl]-isobutylamide was dissolved in THF (8 ml) under argon atmosphere. LiAlH₄ (5.2 ml, 1 M in THF) was added at RT. The reaction mixture was stirred for 2 h at 45° C. After complete reaction the resin was washed twice with THF (5 ml) and then suspended in THF and washed with sat. rochelle's solution. After that the resin was washed 10 times with DMF and CH₂Cl₂ and dried under reduced pressure.

The resin bound to N,N′-diethyl-N-isobutyl-ethylenediamine was suspended in HFIP/CH₂Cl₂ solution (30%, 10 ml) for 10 min. This procedure was repeated twice. The solvents from the combined organic solution were removed under reduced pressure. The residue was transferred to a CH₂Cl₂ solution containing HCl (0.1 ml HCl in dioxane, 4 M in 2 ml CH₂Cl₂) and the solvent was removed again. The resulting N,N′-diethyl-N-isobutyl-ethylenediamine (208 mg, 1 mmol, 77% referred to 1.3 mmol resin) was used without any further purification in THF/CH₂Cl₂ (1:1, 1 ml) for further use.

8 (1 eq, 1.00 g, 1.08 mmol) was dissolved in dry THF (10 ml) under an argon atmosphere and p-nitrophenylchloroformate (0.55 g, 2.70 mmol, 2.5 eq) and DIPEA (0.77 ml, 4.32 mmol, 4 eq) were added. The reaction mixture was stirred for 1 h at RT and then quenched with 1 ml AcOH. The solvent was removed and the residue was purified using the automated Flash chromatography (Cartridge; SNAP 50 g, solvent A: heptanes, solvent B: EtOAc, 10-54% B over 13 CV).

p-nitrophenylcarbonate: Yield 0.812 g (0.745 mmol, 69%)

MS [M+Na]⁺=1111.43 g/mol (MW+Na calculated=1111.43 g/mol).

The p-nitrophenylcarbonate (0.376 g, 0.345 mmol) was dissolved in THF under a nitrogen atmosphere and N,N′-diethyl-N-isobutyl-ethylenediamine (0.18 g, 0.86 mmol, 2.5 eq) and DIPEA (0.246 ml, 1.38 mmol, 4 eq) were added. The reaction mixture was stirred for 30 min at RT and then quenched with 1 ml AcOH. The solvents were removed and the residue was purified by RP-HPLC and lyophilized.

9a: Yield 158 mg (0.14 mmol, 41%).

MS [M+H]⁺=1123.61 g/mol (MW calculated=1122.64 g/mol).

9b was synthesized as described above except for the use of diethylamine instead of N,N′-diethyl, N-isobutyl-ethylenediamine. The p-nitrophenylcarbonate was not purified and used in situ to obtain 9b.

9b: Yield 755 mg (0.44 mmol, 76%).

MS [M+H]⁺=1023.46 g/mol (MW calculated=1022.51 g/mol).

Synthesis of 10a

To a RT solution of 9a*HCl (0.302 g, 0.27 mmol) in THF/MeOH 2:1 (12 ml) were added 3 drops of a sat. aqueous NaHCO₃ solution to adjust the pH to 5.0. NaBH₄ (0.104 g, 2.77 mmol) was added in small portions, and the mixture was stirred at RT for 10 min. After addition of HOAc (0.63 ml), the reaction mixture was partitioned between 25 ml CH₂Cl₂ and water (25 ml) and brine (25 ml). The aqueous phase was extracted with CH₂Cl₂ (4×50 ml), and the combined organic fractions were dried over MgSO₄, filtered and concentrated. The crude material was purified by automated flash chromatography on silica (SNAP 50 g cartridge, flow 40 ml/min, solvent A: EtOAc, solvent B: 0.02% EtNMe₂ in CH₂Cl₂, solvent C: 0.02% EtNMe₂ in MeOH; gradient 100% A (7.6 CV), 0-100% B in A (1.0 CV), 100% B (1.0 CV), 5% C in B (2.6 CV), 11% C in B (2.4 CV), 17% C in B (6.3 CV)).

10a: Yield 0.14 g (0.124 mmol, 46%) as white solid.

MS [M+H]⁺=1124.55 (MW calculated=1123.51 g/mol).

10b was synthesized as described above except for the use of 9b instead of 9a and purification using the automated Flash chromatography system.

10 b: Yield 0.534 g (5.2 mmol, 71%).

MS [M+H]⁺=1025.52 g/mol (MW+H calculated=1024.6 g/mol)

Synthesis of 11a

Benzyl alcohol 10a (140 mg, 0.136 mmol) was dissolved in dry MeCN (10 ml) and the solvent was evaporated at room temperature in vacuo. Under a nitrogen atmosphere the residue was redissolved in dry MeCN (10 mL) and bis-pentafluorophenyl-carbonate (2.5 eq., 134 mg, 0.34 mmol), DMAP (2 mg, 16 μmol), and DIPEA (5 eq., 120 μl, 0.68 mmol) were added. The reaction mixture was stirred for 10 min at room temperature, cooled to −18° C., and acidified with AcOH (0.1 ml). The solvents were removed under reduced pressure and 11a was purified by RP-HPLC and lyophilized at 0-5° C.

11a: Yield 94 mg (52%)

MS [M+H]⁺=1334.61 g/mol (MW+H calculated=1334.70 g/mol)

11b was synthesized as described above except for the use of 10b instead of 10a.

11b: Yield 391 mg (0.31 mmol, 61%).

MS [M+H]⁺=1234.45 g/mol (MW+H calculated=1234.50 g/mol).

### Example 2

### Synthesis of Permanent and Transient PEG-Linker Reagent 13a and 13b

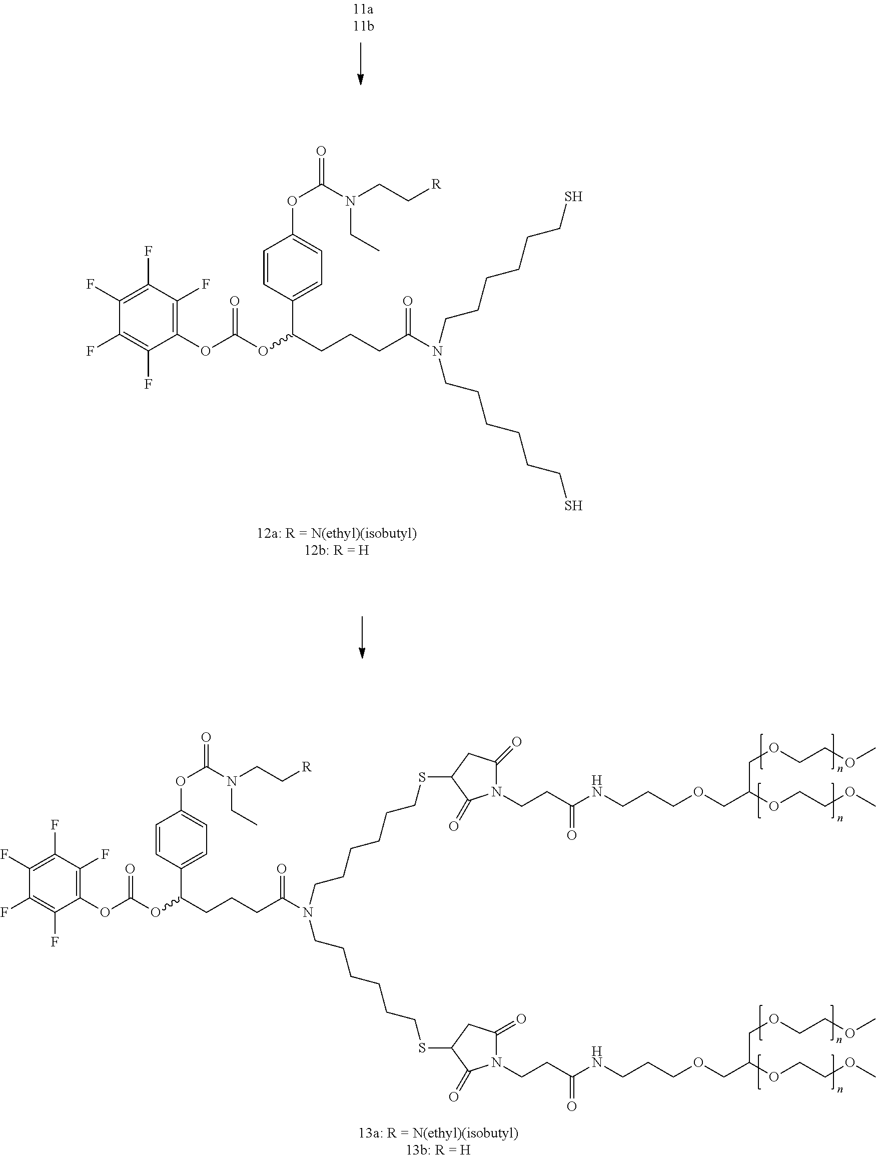

Carbonate 11a (20 mg, 15 μmol) was cooled down in a N₂-bath under argon atmosphere. AcOH (9 μl) and HFIP (470 μl) were added and the reaction mixture stirred at RT until all solids were dissolved. Then the reaction mixture was cooled down again and TES (9 μl) was added and the solution was stirred at 0° C. until complete decolorization. The reaction mixture was diluted with 1.5 ml MeCN/H₂O (9:1, 0.05% TFA) and purified by RP-HPLC.

12a: Yield 5.2 mg (6 μmol, 42%)

MS [M+H]⁺=851.10 g/mol (MW+H calculated=851.07 g/mol).

12b was prepared accordingly from 11b (60 mg, 49 μmol)

12b: Yield 8 mg (10.6 μmol, 22%).

MS [M+H]⁺=751.28 g/mol (MW+H calculated=751.30 g/mol).

mPEG2×20kDa-maleimide (NOF, Japan) (521 mg, 12.7 μmol) was added to 5.2 mg (6 μmol) 12a in 6 mL 3/1 (v/v) MeCN/H₂O+0.1% TFA. 297 μl of 0.5 M phosphate buffer pH 7.4 were added and the mixture was reacted for 10 min at RT. 1.5 μl (13 μmol) mercaptoethanol was added and the reaction mixture was acidified to pH 4-5 by addition of TFA. 13a was purified by RP-HPLC and lyophilized.

13a: Yield 319 mg (pfp-carbonate activity 83%).

13b was synthesized as described for 13a except for using 12b (8 mg, 15.6 μmol) instead of and mPEG2×20kDa-maleimide 12a (1.65 g, 41 μmol).

13b: Yield 933 mg (pfp-carbonate activity 71%).

### Example 3

### Synthesis of Permanent Carbamate-Linked mPEG-IFN-2a Monoconjugate 14 Using 4-Arm Branched 80 kDa mPEG-pentafluorophenylcarbonate Derivative 13b

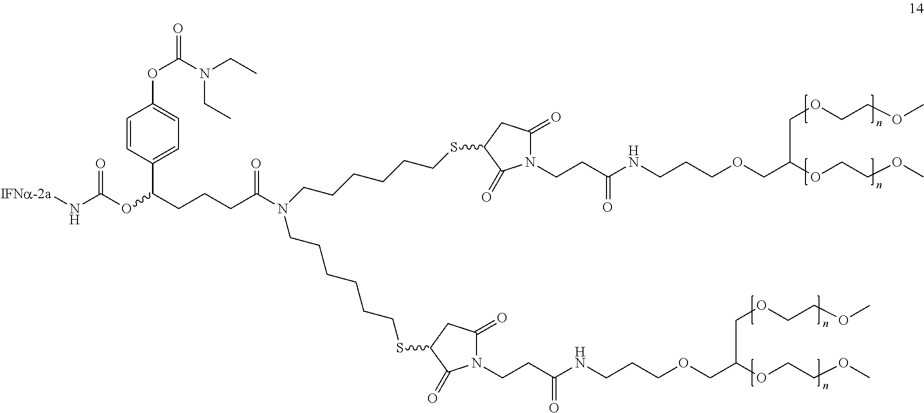

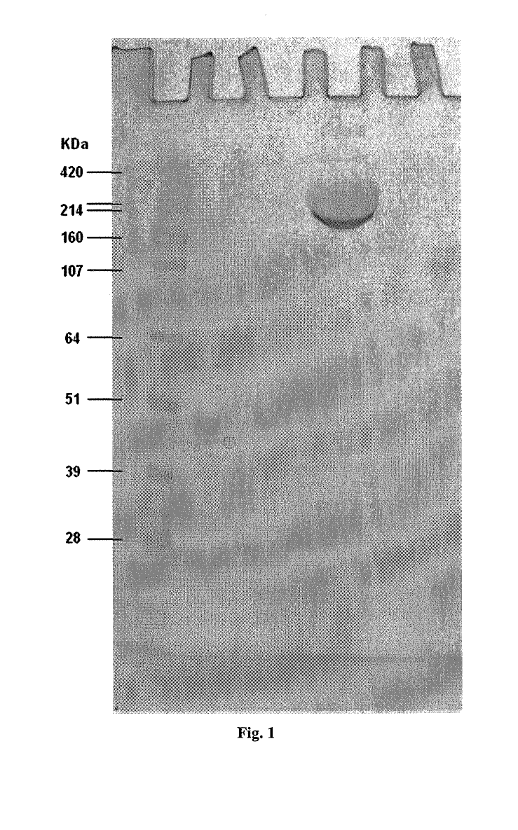

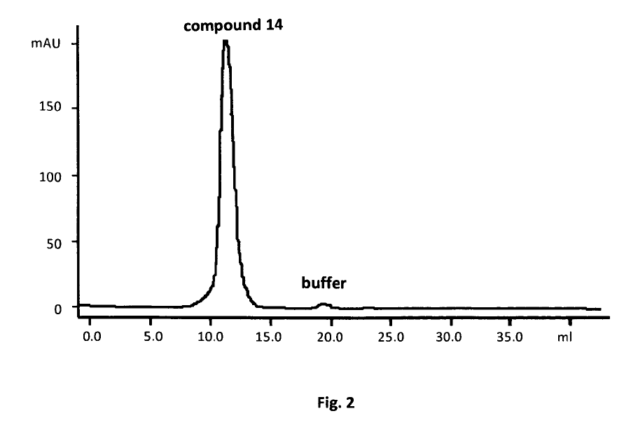

IFN-2a was buffer exchanged to 50 mM sodium borate pH 9 (alternatively sodium borate pH 8.5 or sodium borate pH 8 can be used) and chilled to 4° C. The concentration of IFN-2a was approximately 5 mg/ml. A five-fold molar excess of permanent 4-arm branched 80 kDa mPEG-linker reagent 13b relative to the amount of IFN-2a was dissolved on an ice-bath in water to form a 20% (w/v) reagent solution. The reagent solution was added to the IFN-2a solution and gently mixed. The reaction mixture was incubated for 6 h at 4° C. and quenched by incubating in 100 mM hydroxylamine at pH 7 and RT for 2 h. Permanent mPEG-linker-IFN-2a monoconjugate 14 was purified by cation exchange chromatography at pH 4 and analyzed by SDS-PAGE (see FIG. 1) and size exclusion chromatography (see FIG. 2).

### Example 4

### Synthesis of Permanent Carbamate-Linked mPEG-IFN-2b Monoconjugate 15 Using 4-Arm Branched 80 kDa mPEG-pentafluorophenylcarbonate Derivative 13b

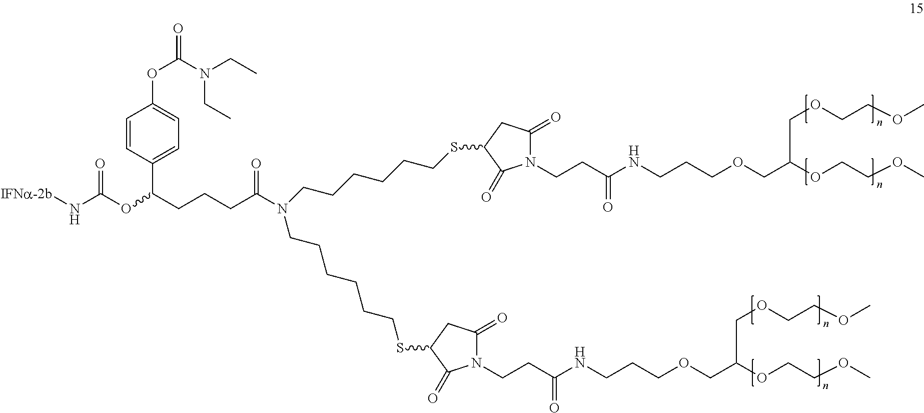

Permanent carbamate-linked mPEG-IFN-2b monoconjugate 15 was synthesized according to Example 3 using IFN-2b and 4-arm branched 80kDa mPEG-pentafluorophenyl carbonate derivative 13b.

### Example 5

### Synthesis of Polymeric Carrier Linked Prodrug 16 Using 4-Arm Branched 80 kDa mPEG-pentafluorophenylcarbonate Derivative 13a

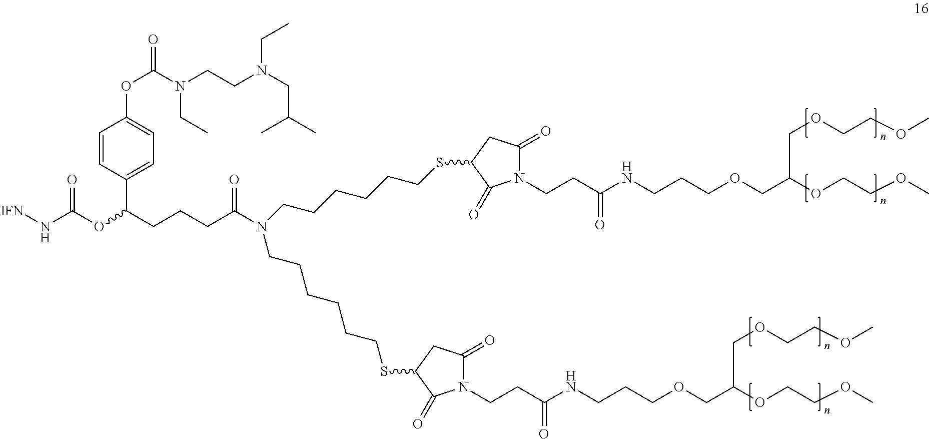

Transient carbamate-linked mPEG-IFN-2a monoconjugate 16 was synthesized according to Example 3 using IFN-2a and 4-arm branched 80 kDa mPEG-pentafluorophenyl carbonate derivative 13a.

### Example 6

### Assay to Measure In Vitro Antiviral Activity of Interferon and In Vitro Antiviral Residual Activity of Permanent PEG Interferon Conjugates

The antiviral potency of interferon-2a, interferon-2b and the corresponding non-cleavable PEG-interferon conjugates were determined in a cell based in vitro assay according to the European Pharmacopoeia. This cell based anti-viral assay determines the relative potency which is calibrated in International Units. The basis of this assay is the inhibitory effect that interferons exhibit on cells to prevent them from viral infection. For the detection and quantification of the viral cytopathic effect, a colorimetric assay for the quantification of cell proliferation and cell viability is used, In this assay, the tetrazolium salt WST-1 is metabolized by mitochondrial dehydrogenases of living cells and results in a color change. The assay was performed with human Hep-2C cells and cytopathogenic encephalomyocarditis virus (EMCV) as the challenge virus for the antiv-viral state of the inoculated cells.

The antiviral potencies of the conjugates 14 and 15 were determined to be less than 1% of the unconjugated interferon-2a and interferon-2b, respectively.

### Example 7

### Assay to Measure In Vitro Auto-Cleavage Rate of the Transient Linker of TranCon PEG Interferon Conjugates

Determination of In Vitro Carrier-Linked Prodrug Cleavage Half Life in Buffer

For determination of in vitro linker cleavage rate of PEG-linker-IFN prodrug 16, the compound was dissolved in buffer at pH 7.4 (e.g. 20 mM sodium phosphate, 135 mM NaCl, 3 mM EDTA) and solution was filtered through a 0.22 μm filter and incubated at 37° C. Samples were taken at time intervals and analyzed by size exclusion chromatography at 215 nm using Superdex200 column. Peaks corresponding to liberated IFN are integrated and plotted against incubation time. Curve fitting software is applied to determine first-order cleavage rates. A release half-life of 14 days was determined

Determination of Polymeric Carrier-Linked Prodrug Cleavage Half-Life Under Physiological Conditions in 80% Human Plasma

For determination of in vitro linker cleavage rate of PEG-linker-IFN prodrug 16 in 80% human plasma, the compound was dissolved in 4/1 (v/v) human plasma/50 mM sodium phosphate buffer at pH 7.4 and solution was filtered through a 0.22 μm filter and incubated at 37° C. Samples were taken at time intervals and analyzed by an ELISA (e.g. VeriKine™ Human IFN-Alpha Serum Sample ELISA, PBL Interferonsource, USA). This linker cleavage determination using an ELISA is based on the fact, that PEG-linker-IFN conjugates show lower signals in an ELISA as compared to free IFN at the same concentration due to the shielding of the IFN by the conjugated PEG moieties against the antibodies used in the ELISA. Liberated IFN was determined based on the increase of the ELISA signal over time and a calibration curve using unconjugated IFN and the amount of liberated free IFN was plotted against incubation time. Curve fitting software was applied to determine first-order cleavage rates. A release half-life of approximately 12 days was determined.

### Example 8

### Pharmacodynamic Analysis in Cynomolgus Monkeys

Animal Studies were Performed by MPI Research, Inc. (Mattawan/MI, USA).

12 female cynomolgus monkeys (approximately 3.0 kg±0.3 kg) were transferred from a stock colony, placed on study, and assigned to three treatment groups (4 animals/group). The animals were fasted overnight prior to dosing and food was withheld through the first 3 hours of blood sample collection. Total fasting time did not exceed 24 hours.

PEGIntron (Schering-Plough) was administered to Group 1 animals via a single subcutaneous (SC) dose at a dose level of 0.2 mg/kg. PEGylated interferon alpha 16 was administered to Group 2 and 3 animals via a single SC dose at a dose level of 0.5 mg/kg and 1.0 mg/kg, respectively. The doses were administered via bolus injection between the skin and underlying layers of tissue in the scapular region on the back of each animal.

Blood samples (approximately 1.0 ml) were collected from the femoral artery/vein at various time points (approx. 85 min before drug application and at times 1, 3, 6, 12, 24, 36, 48, 72, 96, 120, 144, 168, 192, 216, 240, 264, 288, 312, 336, 360, 384, 408, 432, 456, 480, 528, 576, 624, and 672 h after drug application) and stored at room temperature. Samples were collected into tubes containing no anticoagulant. The samples were allowed to clot for at least 30 minutes until placed on ice. The samples were centrifuged under refrigerated conditions following completion of sample collection at each interval. The resulting serum was separated into six aliquots (approximately 75 μl each) and stored frozen until analysis.

Determination of 2′,5′-Oligoadenylate synthetase (2′5′-OAS) activity was performed based on the Eiken (Tokyo, Japan) 2-5A radioimmuno assay kit, distributed by ALPCO Diagnostics (Salem, N.H., USA), catalog number 01-I-AP75.

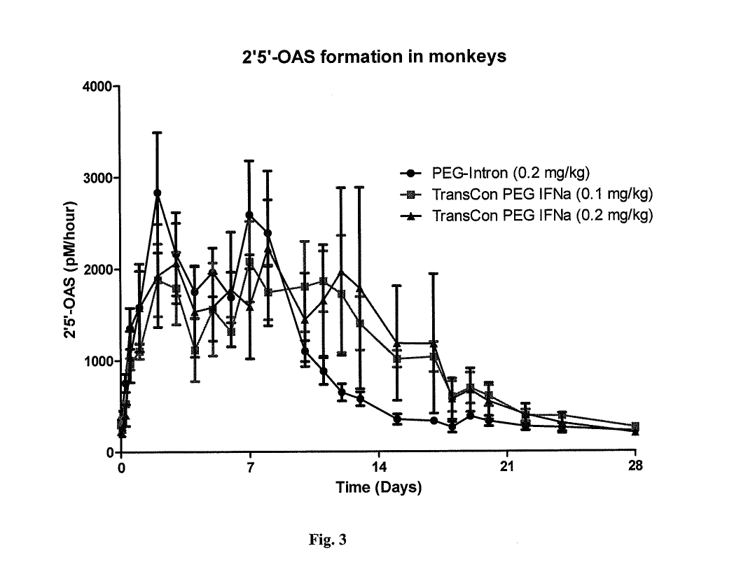

50 μl of sample serum was mixed with 50 μl of poly(I)poly(C) agarose gel solution (catalog number R62301872), vigorously mixed by vortexing and then incubated for 10 min at RT. After adding 1 ml of working buffer (catalog number R6201701+50 μl mercaptoethanol), samples were vortexed for 1 min and centrifuged for 10 min at 2000 rpm at RT. Then, 500 μl of ATP solution (catalog number R6201841 plus 25 ml working buffer/vial) were added, samples were vortexed for 30 sec and incubated at 37° C. for 3 h. To the mixture was added 100 μl of ¹²⁵I-labeled 2-5A solution (catalog number R6021201 plus 5.4 ml ultrapure water/vial) and it was incubated at 37° C. for 1 h and centrifuged at 3200 rpm and 5° C. for 30 min After removing the supernatant, the tubes were placed in a Cobra II Gamma Counter (Packard) and measured with 2 min counting time. The amounts of 2-5A synthesized by 2′5′-OAS were calculated from the standard curve made from the standards provided in the kit. Results are shown in FIG. 3.

### ABBREVIATIONS

2′5′-OAS 2′,5′-Oligoadenylate synthetase

AcOH acetic acid

ATP adenosine triphosphate

CDI carbonyldiimidazole

CV column volume

DBU 1,3-diazabicyclo[5.4.0]undecene

DCM dichloromethane

DIPEA diisopropylethylamine

DMAP dimethylamino-pyridine

DMF N,N-dimethylformamide

DMSO dimethylsulfoxide

EtOH ethanol

EtOAc ethyl acetate

eq stoichiometric equivalent

HFIP hexafluoroisopropanol

HOSu N-hydroxysuccinimide

LCMS mass spectrometry-coupled liquid chromatography

MeCN acetonitrile

MS mass spectrum

MW molecular mass

RP-HPLC reversed-phase high performance liquid chromatography

R_{f} retention factor

RT room temperature

SC subcutaneous

t_{R} retention time

TES triethylsilane

TFA trifluoroacetic acid

THF tetrahydrofurane

Trt trityl

UPLC Ultra-performance liquid chromatography

1. A pharmaceutical composition comprising a water-soluble polymeric carrier linked prodrug of interferon alpha, wherein the prodrug is capable of releasing free interferon alpha, wherein the release half life under physiological conditions is at least 4 days.

2. The composition of claim 1, wherein the release half life is at least 5 days.

3. The composition of claim 1, wherein the molecular weight of the polymeric carrier is in the range of from 40 kDa to 200 kDa.

4. The composition of claim 1, wherein the polymeric carrier is in the range of from 40 kDa to 120 kDa.

5. The composition of claim 1, wherein the interferon alpha is transiently linked to the polymeric carrier such that the release of free interferon alpha is effected through auto-cleavage of an auto-cleavable functional group or linker.

6. The composition of claim 1, wherein the auto-cleavable functional group forms together with a primary amino group of interferon alpha a carbamate or amide group.

7. The composition of claim 1, wherein the prodrug is represented by formula (AA)
IFN-NH-L^{a}-S⁰ (AA),
wherein
IFN-NH represents the interferon alpha residue;
L^{a} represents a functional group, which is auto-cleavable by an auto-cleavage inducing group G^{a};
S⁰ is a branched polymer chain comprising the auto-cleavage inducing group G^{a},
and wherein the molecular weight of the prodrug without the IFN-NH is at least 40 kDa and at most 200 kDa.

8. The composition of claim 7, wherein S⁰ is a polymer chain having a molecular weight of at least 5 kDa and comprising an at least first branching structure BS¹, the at least first branching structure BS¹ comprising an at least second polymer chain S¹ having a molecular weight of at least 4 kDa and wherein the molecular weight of the prodrug without the IFN-NH is at least 40 kDa and at most 200 kDa, and wherein at least one of S⁰, BS¹, S¹ further comprises the auto-cleavage inducing group G^{a}.

9. The composition of claim 8, wherein the branching structure BS¹ further comprises an at least third polymer chain S² having a molecular weight of at least 4 kDa or at least one of S⁰, S¹ comprises an at least second branching structure BS² comprising the at least third polymer chain S having a molecular weight of at least 4 kDa and wherein the molecular weight of the prodrug without the IFN-NH is at least 40 kDa and at most 200 kDa, and wherein at least one of S⁰, BS¹, BS², S¹, S² further comprises the auto-cleavage inducing group G^{a}.

10. The composition of claim 9, wherein the molecular weight of the prodrug without the IFN-NH residue is at least 40 kDa and at most 120 kDa.

11. The composition of claim 10, wherein L^{a} is selected from the group consisting of C(O)—O—, and C(O)—, which form together with the primary amino group of IFN a carbamate or amide group resulting in formula (AA1) or (AA2)
IFN-NH—C(O)O—S⁰ (AA1),
IFN-NH—C(O)—S⁰ (AA2).

12. The composition of claim 11, wherein L^{a} forms together with the amino group of interferon alpha a carbamate functional group, the cleavage of said group is induced by a hydroxyl or amino group of G^{a} via 1,4- or 1,6 benzyl elimination of S⁰, wherein G^{a} contains ester, carbonate, carbamate, or amide bonds that undergo rate-limiting transformation.

13. The composition of claim 12, wherein at least one of the branching structures BS¹, BS² comprises a further fourth polymer chain S³ having a molecular weight of at least 4 kDa or one of S⁰, S¹, S² comprises a third branching structure BS³ comprising the at least fourth polymer chain S³ having a molecular weight of at least 4 kDa and wherein at least one of S⁰, BS¹, BS², BS³, S¹, S₂, S³ further comprises the auto-cleavage inducing group G^{a}.

14. The composition of claim 13, wherein the two or more chains S⁰, S¹, S², S³ are independently based on a polymer selected from the group consisting of polyalkoxy polymers, hyaluronic acid and derivatives thereof, hydroxyalkyl starch and derivatives thereof, polyvinyl alcohols, polyoxazolines, polyanhydrides, poly(ortho)esters, polycarbonates, polyurethanes, polyacrylic acids, polyacrylamides, polyacrylates, polymethacrylates, polyorganophosphazenes, polysiloxanes, polyvinylpyrrolidone, polycyanoacrylates, polyamides and polyesters and corresponding block copolymers.

15. The composition of claim 14, wherein the at least two or more chains S⁰, S¹, S², S³ are based on a polyalkoxy polymer.

16. The composition of claim 15, wherein the shortest distance between the attachment site of S⁰ to L^{a} and the first branching structure BS¹ measured as connected atoms is less than 50 atoms.

17. The composition of claim 16, wherein the shortest distance is less than 20 atoms.

18. The composition of claim 17, wherein S⁰ is of formula (AAA1) 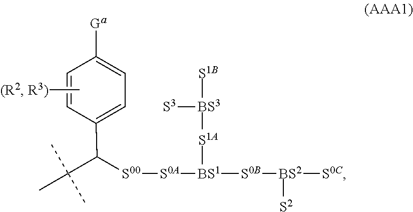
wherein
G^{a} has the meaning as indicated in claim 7;
S⁰⁰ is CH₂; or C(O);
S^{0A} is an alkylene chain having less than 40 carbon atoms, which is optionally interrupted or terminated by one or more groups, cycles or heteroatoms selected from the group consisting of optionally substituted heterocycle; O; S; C(O); and NH; BS¹, BS², BS³ are independently selected from the group consisting of N; and CH;
S^{0B}, S^{1A} are independently an alkylene chain having from 1 to 25 carbon atoms, which is optionally interrupted or terminated by one or more groups, cycles or heteroatoms selected from the group consisting of optionally substituted heterocycle; O; S; C(O); and NH;
S^{0C}, S^{1B}, are (C(O))ₙ₂(CH₂)ₙ₁(OCH₂CH₂)ₙOCH₃, wherein each n is independently an integer from 90 to 2500, each n1 is independently an integer from 1 to 25 and n2 is 0; or 1
S², S³ are independently hydrogen; or (C(O))ₙ₂(CH₂)ₙ₁(OCH₂CH₂)ₙOCH₃, wherein each n is independently an integer from 90 to 2500, each n1 is independently an integer from 1 to 25, and n2 is 0; or 1;
R², R³ are independently selected from the group consisting of hydrogen; methyl; ethyl; propyl; isopropyl; butyl; isobutyl; and tert-butyl.

19. The composition of claim 18, wherein G^{a} is OC(O)—R and R is the partial structure of formula (I) 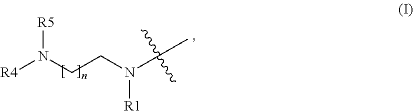
wherein R1, R4, R5 are independently selected from the group consisting of hydrogen; methyl; ethyl; propyl; isopropyl; butyl; isobutyl; and tert-butyl, and wherein n is 1 or 2.

20. The composition of claim 17, wherein L^{a}—S⁰ is represented by formula (AAA2), 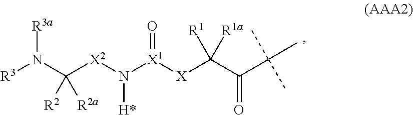
wherein the dashed line indicates the attachment to the primary amino group of IFN so that L^{a} and the amino group form an amide bond;
X is C(R⁴R^{4a}); N(R⁴); O; C(R⁴R^{4a})—C(R⁵R^{5a}); C(R⁵R^{5a})—C(R⁴R^{4a}); C(R⁴R^{4a})—N(R⁶); N(R⁶)—C(R⁴R^{4a}); C(R⁴R^{4a})—O; or O—C(R⁴R^{4a});
X¹ is C; or S(O);
X² is C(R⁷, R^{7a}); or C(R⁷, R^{7a})—C(R⁸, R^{8a});
X³ is O; S; or N—CN;
R¹, R^{1a}, R², R^{2a}, R³, R^{3a}, R⁴, R^{4a}, R⁵, R^{5a}, R⁶, R⁷, R^{7a}, R⁸, R^{8a} are independently selected from the group consisting of H; and C₁₋₄ alkyl;
Optionally, one or more of the pairs R^{1a}/R^{4a}, R^{1a}/R^{5a}, R^{4a}/R^{5a}, R^{7a}/R^{8a} form a chemical bond;
Optionally, one or more of the pairs R¹/R^{1a}, R²/R^{2a}, R⁴/R^{4a}, R⁵/R^{5a}, R⁷/R^{7a}, R⁸R^{8a} are joined together with the atom to which they are attached to form a C₃₋₇ cycloalkyl; or 4 to 7 membered heterocyclyl;
Optionally, one or more of the pairs R¹/R⁴, R¹/R⁵, R¹/R⁶, R⁷/R⁸, R²/R³ are joined together with the atoms to which they are attached to form a ring A;
Optionally, R³/R^{3a} are joined together with the nitrogen atom to which they are attached to form a 4 to 7 membered heterocycle;
A is selected from the group consisting of phenyl; naphthyl; indenyl; indanyl; tetralinyl; C₃₋₁₀ cycloalkyl; 4 to 7 membered heterocyclyl; and 9 to 11 membered heterobicyclyl; and
wherein S⁰ is substituted with one group L²-Z and optionally further substituted, provided that the hydrogen marked with the asterisk in formula (I) is not replaced by a substituent; wherein
L² is a single chemical bond or a spacer; and
Z is of formula (AAA2a) 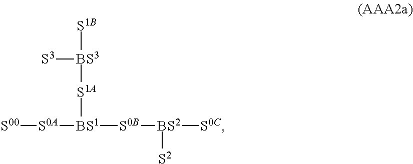
wherein S⁰⁰, S^{0A}, S^{0B}, S^{0C}, S^{1A}, S^{1B}, S², S³, BS¹, BS², and BS³ have the meaning as indicated for formula (AAA1) in claim 18.

21. A composition of claim 1, wherein the prodrug is represented by formula (AB)
IFN-(NH-L-S⁰)ₙ (AB),
wherein
n is 2, 3, or 4;
IFN(-NH)ₙ represents the interferon alpha
each L is independently a permanent functional group L^{p}; or a functional group L^{a}, which is auto-cleavable by an auto-cleavage inducing group G^{a}; and
each S⁰ is independently a polymer chain having a molecular weight of at least 5 kDa, wherein S⁰ is optionally branched by comprising an at least first branching structure BS¹, the at least first branching structure BS¹ comprising an at least second polymer chain S¹ having a molecular weight of at least 4 kDa, wherein at least one of S⁰, BS¹, S¹ further comprises the auto-cleavage inducing group G^{a} and wherein the molecular weight of the prodrug without the IFN-NH is at least 20 kDa and at most 400 kDa.

22. The composition of claim 21, wherein n is 2.

23. The composition of claim 22, wherein the prodrug has a residual activity in an in vitro antiviral assay of less than 5%.

24. The composition of claim 21 which is a water-soluble polymeric carrier linked prodrug of interferon alpha.

25. A method for treating, controlling, delaying or preventing in a mammalian patient in need of the treatment of a condition that can benefit from interferon alpha treatment, wherein the method comprises the administration to said patient a therapeutically effective amount of the pharmaceutical composition of claim 1.

26. The method of claim 25, wherein the prodrug is represented by formula (AB)
IFN-(NH-L-S⁰)ₙ (AB),
wherein
n is 2, 3, or 4;
IFN(-NH)ₙ represents the interferon alpha
each L is independently a permanent functional group L^{p}; or a functional group L^{a}, which is auto-cleavable by an auto-cleavage inducing group G^{a}; and
each S⁰ is independently a polymer chain having a molecular weight of at least 5 kDa, wherein S⁰ is optionally branched by comprising an at least first branching structure BS¹, the at least first branching structure BS¹ comprising an at least second polymer chain S¹ having a molecular weight of at least 4 kDa, wherein at least one of S⁰, BS¹, S¹ further comprises the auto-cleavage inducing group G^{a} and wherein the molecular weight of the prodrug without the IFN-NH is at least 20 kDa and at most 400 kDa.

27. The method of claim 25, wherein the patient is virally infected and the treatment of the virally infected patient results in a reduced viral relapse rate compared to a permanently linked PEGylated interferon alpha conjugate.

28. The method of claim 27, wherein the administration results in an increased volume of distribution over permanently linked PEGylated interferon alpha conjugate.

29. The pharmaceutical composition according to claim 1, wherein the pharmaceutical composition is dry.

30. The pharmaceutical composition according to claim 29, wherein the pharmaceutical composition was dried by lyophilization.

31. A pharmaceutical composition according to claim 1, wherein the pharmaceutical composition is liquid.

32. The pharmaceutical composition according to claim 1, wherein the polymeric carrier-linked interferon alpha prodrug is sufficiently dosed in the composition to provide a therapeutically effective amount of interferon alpha for one week or longer in one application.

33. The pharmaceutical composition according to claim 1, wherein it is a single dose composition.

34. The pharmaceutical composition according to claim 1, wherein it is a multiple dose composition.

35. A container comprising the pharmaceutical composition according to claims 1.

36. The container of claim 35, wherein the container is a dual-chamber syringe.

37. A method of preparing a reconstituted composition from the dry compositions according to claim 29, comprising the steps of reconstituting the dry pharmaceutical composition by adding reconstitution solution.

38. A method of preparing a liquid composition according to claim 31, comprising the steps of
(i) admixing the polymeric carrier-linked interferon alpha prodrug with one or more excipients,
(ii) transfering amounts equivalent to single or multiple doses into a suitable container, and (iii) sealing the container.

39. A method of preparing a dry composition according claim 29, comprising the steps of (i) admixing the polymeric carrier-linked interferon alpha prodrug with one or more excipients, (ii) transfering amounts equivalent to single or multiple doses into a suitable container,
(iii) drying the composition in said container, and (iv) sealing the container.

40. A kit of parts, comprising a needle and a container for use with the needle and wherein such container comprises the liquid composition according to claim 31.

41. A kit of parts, comprising a syringe, a needle and a first container comprising the dry polymeric carrier-linked interferon alpha prodrug composition according to claim 29 for use with the syringe and a second container comprising the reconstitution solution.

42. A kit of parts according to claim 41, wherein the first and second container form a dual-chamber syringe and wherein one of the two chambers of the dual-chamber syringe contains the dry pharmaceutical composition and the second chamber of said dual-chamber syringe contains the reconstitution solution.
